(19) United States
(12) Patent Application Publication
(10) Pub. No.: US 2021/0005224 A1
(43) Pub. Date: Jan. 7, 2021

(54) System and Method for Determining a State of a User

(71) Applicants: Richard A. Rothschild, London (GB); Robin S. Slomkowski, Eugene, OR (US)

(72) Inventors: Richard A. Rothschild, London (GB); Robin S. Slomkowski, Eugene, OR (US)

(21) Appl. No.: 16/898,435

(22) Filed: Jun. 11, 2020

Related U.S. Application Data
(63) Continuation-in-part of application No. 15/256,543, filed on Sep. 3, 2016.
Application No. 15/256,543 is a continuation-in-part of application No. 16/704,844, filed on Dec. 5, 2019.
Application No. 16/704,844 is a continuation of application No. 16/273,141, filed on Feb. 11, 2019, now Pat. No. 10,522,188.
Application No. 16/273,141 is a continuation of application No. 15/495,485, filed on Apr. 24, 2017, now Pat. No. 10,242,713.
Application No. 15/495,485 is a continuation of application No. 15/293,211, filed on Oct. 13, 2016, now abandoned.
(60) Provisional application No. 62/214,496, filed on Sep. 4, 2015.
(60) Provisional application No. 62/240,783, filed on Oct. 13, 2015.

(51) Int. Cl. G11B 27/10; G11B 27/031; G06K 9/00; H04N 5/76; H04N 9/82; G16H 40/63

(52) U.S. Cl. CPC G11B 27/10; G11B 27/031; G06K 9/00892; G06K 2009/00939; H04N 9/8205; G11B 27/102; G16H 40/63; H04N 5/76

(57) ABSTRACT

A method is provided for using at least self-reporting and biometric data to determine a current state of a user. The method includes receiving first biometric data of the user (e.g., using a camera on a mobile device) at a first period of time and self-reporting data shortly thereafter, where the first biometric data comprises at least changes in the user's pupil in response to first visuals (e.g., a series of different light intensities, etc.) (e.g., provided using a display on the mobile device) and the self-reporting data comprises a state of the user, where the self-reporting data is linked to the first biometric data. The method further includes receiving second biometric data at a second time and using the same, along with at least the first biometric data and self-reporting data, to determine (e.g., via AI, manually, etc.) a state of the user at the second period of time.

### BACKGROUND OF THE INVENTION

### 1. Field of the Invention

The present invention relates to determining a current state of a user, and more particularly, to a system and method for using at least self-reporting and biometric data to determine a current state of a user and to perform at least one action in response thereto.

### 2. Description of Related Art

Recently, devices have been developed that are capable of measuring, sensing, or estimating in a convenient form factor at least one or more metric related to physiological characteristics, commonly referred to as biometric data. For example, devices that resemble watches have been developed which are capable of measuring an individual's heart rate or pulse, and, using that data together with other information (e.g., the individual's age, weight, etc.), to calculate a resultant, such as the total calories burned by the individual in a given day. Similar devices have been developed for measuring, sensing, or estimating other kinds of metrics, such as blood pressure, breathing patterns, breath composition, sleep patterns, and blood-alcohol level, to name a few. These devices are generically referred to as biometric devices or biosensor metrics devices.

While the types of biometric devices continue to grow, the way in which biometric data is used remains relatively static. For example, heart rate data is typically used to give an individual information on their pulse and calories burned. By way of another example, blood-alcohol and other data (e.g., eye movement data) is typically used to give an individual information on their blood-alcohol level, and to inform the individual on whether or not they can safely or legally operate a motor vehicle. By way of yet another example, an individual's breathing pattern (measurable for example either by loudness level in decibels, or by variations in decibel level over a time interval) may be monitored by a doctor, nurse, or medical technician to determine whether the individual suffers from sleep apnea.

While biometric data is useful in and of itself, such data would be more informative or dynamic if it could be combined with other data (e.g., video data, etc.), provided (e.g., wirelessly, over a network, etc.) to a remote device, and/or searchable (e.g., allowing certain conditions, such as an elevated heart rate, to be quickly identified) and/or cross-searchable (e.g., using biometric data to identify a video section illustrating a specific characteristic, or vice-versa). Such data would may also indicate how the individual is feeling (e.g., at least one emotional state, mood, physical state, or mental state) at a particular time or in response to the individual being in the presence of at least one thing (e.g., a person, a place, textual content (or words included therein or a subject matter thereof), video content (or a subject matter thereof), audio content (or words included therein or a subject matter thereof), etc.).

Thus, it would be advantageous, and a need exists, for a system and method that uses the determined state (e.g., emotion state, mood, physical state, or mental state), either alone or together with other information (e.g., at least one thing, interest data, at least one request (e.g., question, command, etc.), etc.), to produce a certain result, such as provide the individual with certain web-based content (e.g., a certain web page, a certain advertisement, etc.) and/or perform at least one action. While providing a particular message to every known biometric state may not be reasonable for content creators to understand and target, human emotions and moods provide a specific context for targeting messages that is easily understood by content creators.

A need also exists for an efficient system and method capable of achieving at least some, or indeed all, of the foregoing advantages, and capable of merging the data generated in either automatic or manual form by the various devices, which are often using operating systems or technologies (e.g., hardware platforms, protocols, data types, etc.) that are incompatible with one another.

In certain embodiments of the present invention, the system and/or method is configured to receive, manage, and filter the quantity of information on a timely and cost-effective basis, and could also be of further value through the accurate measurement, visualization (e.g., synchronized visualization, etc.), and rapid notification of data points which are outside (or within) a defined or predefined range.

Such a system and/or method could be used by an individual (e.g., athlete, etc.) or their trainer, coach, etc., to visualize the individual during the performance of an athletic event (e.g., jogging, biking, weightlifting, playing soccer, etc.) in real-time (live) or afterwards, together with the individual's concurrently measured biometric data (e.g., heart rate, etc.), and/or concurrently gathered “self-realization data,” or subject-generated experiential data, where the individual inputs their own subjective physical or mental states during their exercise, fitness or sports activity/training (e.g., feeling the onset of an adrenaline “rush” or endorphins in the system, feeling tired, “getting a second wind,” etc.). This would allow a person (e.g., the individual, the individual's trainer, a third party, etc.) to monitor/observe physiological and/or subjective psychological characteristics of an individual while watching or reviewing the individual in the performance of an athletic event, or other physical activity. Such inputting of the self-realization data, ca be achieved by various methods, including automatically, time-stamped-in-the-system voice notes, short-form or abbreviation key commands on a smart phone, smart watch, enabled fitness band, or any other system-linked input method which is convenient for the individual to utilize so as not to impede (or as little as possible) the flow and practice by the individual of the activity in progress.

Such a system and/or method would also facilitate, for example, remote observation and diagnosis in telemedicine applications, where there is a need for the medical staff, or monitoring party or parent, to have clear and rapid confirmation of the identity of the patient or infant, as well as their visible physical condition, together with their concurrently generated biometric and/or self-realization data.

Furthermore, the system and/or method should also provide the subject, or monitoring party, with a way of using video indexing to efficiently and intuitively benchmark, map and evaluate the subject's data, both against the subject's own biometric history and/or against other subjects' data samples, or demographic comparables, independently of whichever operating platforms or applications have been used to generate the biometric and video information. By being able to filter/search for particular events (e.g., biometric events, self-realization events, physical events, etc.), the acquired data can be reduced down or edited (e.g., to create a “highlight reel,” etc.) while maintaining synchronization between individual video segments and measured and/or gathered data (e.g., biometric data, self-realization data, GPS data, etc.). Such comprehensive indexing of the events, and with it the ability to perform structured aggregation of the related data (video and other) with (or without) data from other individuals or other relevant sources, can also be utilized to provide richer levels of information using methods of “Big Data” analysis and “Machine Learning,” and adding artificial intelligence (“AI”) for the implementation of recommendations and calls to action.

### SUMMARY OF THE INVENTION

The present invention provides (in first part) a system and method for using, processing, indexing, benchmarking, ranking, comparing and displaying biometric data, or a resultant thereof, either alone or together (e.g., in synchronization) with other data (e.g., video data, etc.). Preferred embodiments of the present invention operate in accordance with a computing device (e.g., a smart phone, etc.) in communication with at least one external device (e.g., a biometric device for acquiring biometric data, a video device for acquiring video data, etc.). In a first embodiment of the present invention, video data, which may include audio data, and non-video data, such as biometric data, are stored separately on the computing device and linked to other data, which allows searching and synchronization of the video and non-video data.

The present invention is also directed toward (in second part) personalization preference optimization, or the use of biometric data from an individual to determine at least one emotional state, mood, physical state, or mental state (“state”) of the individual, which is then used, either alone or together with other data (e.g., at least one thing in a proximity of the individual at a time that the individual is experiencing the emotion, interest data from a source of web-based data (e.g., bid data, etc.), etc.) to provide the individual with certain web-based data or to perform a particular action.

With respect to the first part of the present invention, an application (e.g., running on the computing device, etc.) may include a plurality of modules for performing a plurality of functions. For example, the application may include a video capture module for receiving video data from an internal and/or external camera, and a biometric capture module for receiving biometric data from an internal and/or external biometric device. The client platform may also include a user interface module, allowing a user to interact with the platform, a video editing module for editing video data, a file handling module for managing data, a database and sync module for replicating data, an algorithm module for processing received data, a sharing module for sharing and/or storing data, and a central login and ID module for interfacing with third party social media websites, such as Facebook™.

These modules can be used, for example, to start a new session, receive video data for the session (i.e., via the video capture module) and receive biometric data for the session (i.e., via the biometric capture module). This data can be stored in local storage, in a local database, and/or on a remote storage device (e.g., in the company cloud or a third-party cloud service, such as Dropbox™, etc.). In a preferred embodiment, the data is stored so that it is linked to information that (i) identifies the session and (ii) enables synchronization.

For example, video data is preferably linked to at least a start time (e.g., a start time of the session) and an identifier. The identifier may be a single number uniquely identifying the session, or a plurality of numbers (e.g., a plurality of global or universal unique identifiers (GUIDs/UUIDs)), where a first number uniquely identifying the session and a second number uniquely identifies an activity within the session, allowing a session to include a plurality of activities. The identifier may also include a session name and/or a session description. Other information about the video data (e.g., video length, video source, etc.) (i.e., “video metadata”) can also be stored and linked to the video data. Biometric data is preferably linked to at least the start time (e.g., the same start time linked to the video data), the identifier (e.g., the same identifier linked to the video data), and a sample rate, which identifies the rate at which biometric data is received and/or stored.

Once the video and biometric data is stored and linked, algorithms can be used to display the data together. For example, if biometric data is stored at a sample rate of 30 samples per minute (spm), algorithms can be used to display a first biometric value (e.g., below the video data, superimposed over the video data, etc.) at the start of the video clip, a second biometric value two seconds later (two seconds into the video clip), a third biometric value two seconds later (four seconds into the video clip), etc. In alternate embodiments of the present invention, non-video data (e.g., biometric data, self-realization data, etc.) can be stored with a plurality of time-stamps (e.g., individual stamps or offsets for each stored value, or individual sample rates for each data type), which can be used together with the start time to synchronize non-video data to video data.

In one embodiment of the present invention, the biometric device may include a sensor for sensing biometric data, a display for interfacing with the user and displaying various information (e.g., biometric data, set-up data, operation data, such as start, stop, and pause, etc.), a memory for storing the sensed biometric data, a transceiver for communicating with the exemplary computing device, and a processor for operating and/or driving the transceiver, memory, sensor, and display. The exemplary computing device includes a transceiver(1) for receiving biometric data from the exemplary biometric device, a memory for storing the biometric data, a display for interfacing with the user and displaying various information (e.g., biometric data, set-up data, operation data, such as start, stop, and pause, input in-session comments or add voice notes, etc.), a keyboard (or other user input) for receiving user input data, a transceiver(2) for providing the biometric data to the host computing device via the Internet, and a processor for operating and/or driving the transceiver(1), transceiver(2), keyboard, display, and memory.

The keyboard (or other input device) in the computing device, or alternatively the keyboard (or other input device) in the biometric device, may be used to enter self-realization data, or data on how the user is feeling at a particular time. For example, if the user is feeling tired, the user may enter the “T” on the keyboard. If the user is feeling their endorphins kick in, the user may enter the “E” on the keyboard. And if the user is getting their second wind, the user may enter the “S” on the keyboard. Alternatively, to further facilitate operation during the exercise, or sporting activity, short-code key buttons such as “T,” “E,” and “S” can be preassigned, like speed-dial telephone numbers for frequently called contacts on a smart phone, etc., which can be selected manually or using voice recognition. This data (e.g., the entry or its representation) is then stored and linked to either a sample rate (like biometric data) or time-stamp data, which may be a time or an offset to the start time that each button was pressed. This would allow the self-realization data to be synchronized to the video data. It would also allow the self-realization data, like biometric data, to be searched or filtered (e.g., in order to find video corresponding to a particular event, such as when the user started to feel tired, etc.).

In an alternate embodiment of the present invention, the computing device (e.g., a smart phone, etc.) is also in communication with a host computing device via a wide area network (“WAN”), such as the Internet. This embodiment allows the computing device to download the application from the host computing device, offload at least some of the above-identified functions to the host computing device, and store data on the host computing device (e.g., allowing video data, alone or synchronized to non-video data, such as biometric data and self-realization data, to be viewed by another networked device). For example, the software operating on the computing device (e.g., the application, program, etc.) may allow the user to play the video and/or audio data, but not to synchronize the video and/or audio data to the biometric data. This may be because the host computing device is used to store data critical to synchronization (time-stamp index, metadata, biometric data, sample rate, etc.) and/or software operating on the host computing device is necessary for synchronization. By way of another example, the software operating on the computing device may allow the user to play the video and/or audio data, either alone or synchronized with the biometric data, but may not allow the computing device (or may limit the computing device's ability) to search or otherwise extrapolate from, or process the biometric data to identify relevant portions (e.g., which may be used to create a “highlight reel” of the synchronized video/audio/biometric data) or to rank the biometric and/or video data. This may be because the host computing device is used to store data critical to search and/or to rank the biometric data (biometric data, biometric metadata, etc.), and/or software necessary for searching (or performing advanced searching of) and/or ranking (or performing advanced ranking of) the biometric data.

In one embodiment of the present invention, the video data, which may also include audio data, starts at a time “T” and continues for a duration of “n.” The video data is preferably stored in memory (locally and/or remotely) and linked to other data, such as an identifier, start time, and duration. Such data ties the video data to at least a particular session, a particular start time, and identifies the duration of the video included therein. In one embodiment of the present invention, each session can include different activities. For example, a trip to Berlin on a particular day (session) may involve a bike ride through the city (first activity) and a walk through a park (second activity). Thus, the identifier may include both a session identifier, uniquely identifying the session via a globally unique identifier (GUID), and an activity identifier, uniquely identifying the activity via a globally unique identifier (GUID), where the session/activity relationship is that of a parent/child.

In one embodiment of the present invention, the biometric data is stored in memory and linked to the identifier and a sample rate “m.” This allows the biometric data to be linked to video data upon playback. For example, if identifier is one, start time is 1:00 PM, video duration is one minute, and the sample rate is 30 spm, then the playing of the video at 2:00 PM would result in the first biometric value to be displayed (e.g., below the video, over the video, etc.) at 2:00 PM, the second biometric value to be displayed (e.g., below the video, over the video, etc.) two seconds later, and so on until the video ends at 2:01 PM. While self-realization data can be stored like biometric data (e.g., linked to a sample rate), if such data is only received periodically, it may be more advantageous to store this data linked to the identifier and a time-stamp, where “m” is either the time that the self-realization data was received or an offset between this time and the start time (e.g., ten minutes and four seconds after the start time, etc.). By storing video and non-video data separately from one another, data can be easily search and synchronized.

With respect to linking data to an identifier, which may be linked to other data (e.g., start time, sample rate, etc.), if the data is received in real-time, the data can be linked to the identifier(s) for the current session (and/or activity). However, when data is received after the fact (e.g., after a session has ended), there are several ways in which the data can be linked to a particular session and/or activity (or identifier(s) associated therewith). The data can be manually linked (e.g., by the user) or automatically linked via the application. With respect to the latter, this can be accomplished, for example, by comparing the duration of the received data (e.g., the video length) with the duration of the session and/or activity, by assuming that the received data is related to the most recent session and/or activity, or by analyzing data included within the received data. For example, in one embodiment, data included with the received data (e.g., metadata) may identify a time and/or location associated with the data, which can then be used to link the received data to the session and/or activity. In another embodiment, the computing device could display data (e.g., a barcode, such as a QR code, etc.) that identifies the session and/or activity. An external video recorder could record the identifying data (as displayed by the computing device) along with (e.g., before, after, or during) the user and/or his/her surroundings. The application could then search the video data for identifying data, and use this data to link the video data to a session and/or activity. The identifying portion of the video data could then be deleted by the application if desired.

With respect to the second part of the present invention operate, a Web host may be in communication with a plurality of content providers (i.e., sources) and at least one network device via a wide area network (WAN), wherein the network device is operated by an individual and is configured to communicate biometric data of the individual to the Web host. The content providers provide the Web host with content, such as websites, web pages, image data, video data, audio data, advertisements, etc. The Web host is then configured to receive biometric data from the network device, where the biometric data is acquired from and/or associated with an individual that is operating the network device. An application is then used to determine at least one emotion, mood, physical state, or mental state from the received biometric data. This is done using known algorithms and/or correlations between biometric data and various states, as stored in the memory device.

In one embodiment of the present invention, content providers may express interest in providing the web-based data to an individual in a particular emotional state. In another embodiment of the present invention, content providers may express interest in providing the web-based data to an individual or other concerned party (such as friends, employer, care provider, etc.) that experienced a particular emotion in response to a thing (e.g., a person, a place, a subject matter of textual content, a subject matter of video content, a subject matter of audio content, etc.). The interest may be a simple “Yes” or “No,” or may be more complex, like interest on a scale of 1-10, an amount the content owner is willing to pay per impression (CPM), or an amount the content owner is willing to pay per click (CPC).

The interest data, alone or in conjunction with other data (e.g., randomness, demographics, etc.), may be used by the application to determine content data (e.g., an advertisement, etc.) that should be provided to the individual. For example, if the interest data includes different bids for a particular emotion or an emotion-thing relationship, the application may provide the advertisement with the highest bid to the individual that experienced the emotion. In other embodiments, other data is taken into consideration in providing content to the individual. In these embodiments, at least interest data is taken into account in selecting the content that is to be provided to the individual.

In one method of the present invention, biometric data is received from an individual and used to determine a corresponding emotion of the individual, such as happiness, anger, surprise, sadness, disgust, or fear. It is to be understood that emotional categorization is hierarchical and that such a method may allow targeting more specific emotions such as ecstasy, amusement, or relief, which are all subsets of the emotion of joy. A determination is made as to whether the emotion is the individual's current state, or whether it is based on the individual's response to a thing (e.g., a person, place, information displayed to the individual, etc.). If the emotion is the individual's current state, then content is selected based on at least the individual's current emotional state and interest data. If, however, the emotion is the individual's response to a thing, then content is selected based on at least the individual's emotional response to the thing (or subject matter thereof) and interest data. The selected content is then provided to the individual, or network device operated by the individual.

Emotion, mood, physical, or mental state of an individual can also be taken into consideration when performing a particular action or carrying out a particular request (e.g., question, command, etc.). In other words, prior to performing a particular action (e.g., under the direction of an individual, etc.), a network-connected or network-aware system or device may take into consideration an emotion, mood, physical, or mental state of the individual. For example, a command or instruction provided by the individual, either alone or together with other biometric data related to or from the individual, may be analyzed to determinate the individual's current mood, emotional, physical, or mental state. The network-connected or network-aware system or device may then take the individual's state into consideration when carrying out the command or instruction. Depending on the individual's state, the system or device may warn the individual before performing the requested action, or may perform another action, either in additional to or instead of the requested action. For example, if it is determined that a driver of a vehicle is angry or intoxicated, the vehicle may provide the driver with a warning before starting the engine, may limit maximum speed, or may prevent the driver from operating the vehicle (e.g., switch to autonomous mode, etc.).

A more complete understanding of a system and method for using at least self-reporting and biometric data to determine a current state of a user will be afforded to those skilled in the art, as well as a realization of additional advantages and objects thereof, by a consideration of the following detailed description of the preferred embodiment. Reference will be made to the appended sheets of drawings, which will first be described briefly.

### BRIEF DESCRIPTION OF THE DRAWINGS

FIG. 1 illustrates a system for using, processing, and displaying biometric data, and for synchronizing biometric data with other data (e.g., video data, audio data, etc.) in accordance with one embodiment of the present invention;

FIG. 2A illustrates a system for using, processing, and displaying biometric data, and for synchronizing biometric data with other data (e.g., video data, audio data, etc.) in accordance with another embodiment of the present invention;

FIG. 2B illustrates a system for using, processing, and displaying biometric data, and for synchronizing biometric data with other data (e.g., video data, audio data, etc.) in accordance with yet another embodiment of the present invention;

FIG. 3 illustrates an exemplary display of video data synchronized with biometric data in accordance with one embodiment of the present invention;

FIG. 4 illustrates a block diagram for using, processing, and displaying biometric data, and for synchronizing biometric data with other data (e.g., video data, audio data, etc.) in accordance with one embodiment of the present invention;

FIG. 5 illustrates a block diagram for using, processing, and displaying biometric data, and for synchronizing biometric data with other data (e.g., video data, audio data, etc.) in accordance with another embodiment of the present invention;

FIG. 6 illustrates a method for synchronizing video data with biometric data, operating the video data, and searching the biometric data, in accordance with one embodiment of the present invention;

FIG. 7 illustrates an exemplary display of video data synchronized with biometric data in accordance with another embodiment of the present invention;

FIG. 8 illustrates exemplary video data, which is preferably linked to an identifier (ID), a start time (T), and a finish time or duration (n);

FIG. 9 illustrates an exemplary identifier (ID), comprising a session identifier and an activity identifier;

FIG. 10 illustrates exemplary biometric data, which is preferably linked to an identifier (ID), a start time (T), and a sample rate (S);

FIG. 11 illustrates exemplary self-realization data, which is preferably linked to an identifier (ID) and a time (m);

FIG. 12 illustrates how sampled biometric data points can be used to extrapolate other biometric data point in accordance with one embodiment of the present invention;

FIG. 13 illustrates how sampled biometric data points can be used to extrapolate other biometric data points in accordance with another embodiment of the present invention;

FIG. 14 illustrates an example of how a start time and data related thereto (e.g., sample rate, etc.) can be used to synchronized biometric data and self-realization data to video data;

FIG. 15 depicts an exemplary “sign in” screen shot for an application that allows a user to capture at least video and biometric data of the user performing an athletic event (e.g., bike riding, etc.) and to display the video data together (or in synchronization) with the biometric data;

FIG. 16 depict an exemplary “create session” screen shot for the application depicted in FIG. 15, allowing the user to create a new session;

FIG. 17 depicts an exemplary “session name” screen shot for the application depicted in FIG. 15, allowing the user to enter a name for the session;

FIG. 18 depicts an exemplary “session description” screen shot for the application depicted in FIG. 15, allowing the user to enter a description for the session;

FIG. 19 depicts an exemplary “session started” screen shot for the application depicted in FIG. 15, showing the video and biometric data received in real-time;

FIG. 20 depicts an exemplary “review session” screen shot for the application depicted in FIG. 15, allowing the user to playback the session at a later time;

FIG. 21 depicts an exemplary “graph display option” screen shot for the application depicted in FIG. 15, allowing the user to select data (e.g., heart rate data, etc.) to be displayed along with the video data;

FIG. 22 depicts an exemplary “review session” screen shot for the application depicted in FIG. 15, where the video data is displayed together (or in synchronization) with the biometric data;

FIG. 23 depicts an exemplary “map” screen shot for the application depicted in FIG. 15, showing GPS data displayed on a Google map;

FIG. 24 depicts an exemplary “summary” screen shot for the application depicted in FIG. 15, showing a summary of the session;

FIG. 25 depicts an exemplary “biometric search” screen shot for the application depicted in FIG. 15, allowing a user to search the biometric data for particular biometric event (e.g., a particular value, a particular range, etc.);

FIG. 26 depicts an exemplary “first result” screen shot for the application depicted in FIG. 15, showing a first result for the biometric event shown in FIG. 25, together with corresponding video;

FIG. 27 depicts an exemplary “second result” screen shot for the application depicted in FIG. 15, showing a second result for the biometric event shown in FIG. 25, together with corresponding video;

FIG. 28 depicts an exemplary “session search” screen shot for the application depicted in FIG. 15, allowing a user to search for sessions that meet certain criteria;

FIG. 29 depicts an exemplary “list” screen shot for the application depicted in FIG. 15, showing a result for the criteria shown in FIG. 28;

FIG. 30 illustrates a Web host in communication with at least one content provider and at least one network device via a wide area network (WAN), wherein said Web host is configured to provide certain content to the network device in response to biometric data (or data related thereto), as received from the network device;

FIG. 31 illustrates one embodiment of the Web host depicted in FIG. 30;

FIG. 32 provides an exemplary chart that links different biometric data to different emotions;

FIG. 33 provides an exemplary chart that links different responses to different emotions, different things, and different interest levels in the same;

FIG. 34 illustrates a method in accordance with one embodiment of the present invention of using biometric data from an individual to determine at least one emotion of the individual, and using the at least one emotion, either alone or in conjunction with other data, to select content to be provided to the individual;

FIG. 35 provides an exemplary biometric-sensor data string in accordance with one embodiment of the present invention;

FIG. 36 provides an exemplary emotional-response data string in accordance with one embodiment of the present invention;

FIG. 37 provides an exemplary emotion-thing data string in accordance with one embodiment of the present invention;

FIG. 38 provides an exemplary thing data string in accordance with one embodiment of the present invention;

FIG. 39 illustrates a network-enabled device that is in communication with a plurality of remote devices via a wide area network (WAN) and is configured to use biometric data to determine at least one state of an individual and use the at least one state to perform at least one action;

FIG. 40 illustrates one embodiment of the network-enabled device depicted in FIG. 39; and

FIG. 41 illustrates a method in accordance with one embodiment of the present invention of using biometric data from an individual to determine at least one state of the individual, and using the at least one state to perform at least one action.

### DETAILED DESCRIPTION OF THE PREFERRED EMBODIMENT

The present invention (in first part) provides a system and method for using, processing, indexing, benchmarking, ranking, comparing and displaying biometric data, or a resultant thereof, either alone or together (e.g., in synchronization) with other data (e.g., video data, etc.). It should be appreciated that while this part of the invention is described herein in terms of certain biometric data (e.g., heart rate, breathing patterns, blood-alcohol level, etc.), the invention is not so limited, and can be used in conjunction with any biometric and/or physical data, including, but not limited to oxygen levels, CO₂ levels, oxygen saturation, blood pressure, blood glucose, lung function, eye pressure, body and ambient conditions (temperature, humidity, light levels, altitude, and barometric pressure), speed (walking speed, running speed), location and distance travelled, breathing rate, heart rate variance (HRV), EKG data, perspiration levels, calories consumed and/or burnt, ketones, waste discharge content and/or levels, hormone levels, blood content, saliva content, audible levels (e.g., snoring, etc.), mood levels and changes, galvanic skin response, brain waves and/or activity or other neurological measurements, sleep patterns, physical characteristics (e.g., height, weight, eye color, hair color, iris data, fingerprints, etc.) or responses (e.g., facial changes, iris (or pupil) changes, voice (or tone) changes, etc.), or any combination or resultant thereof.

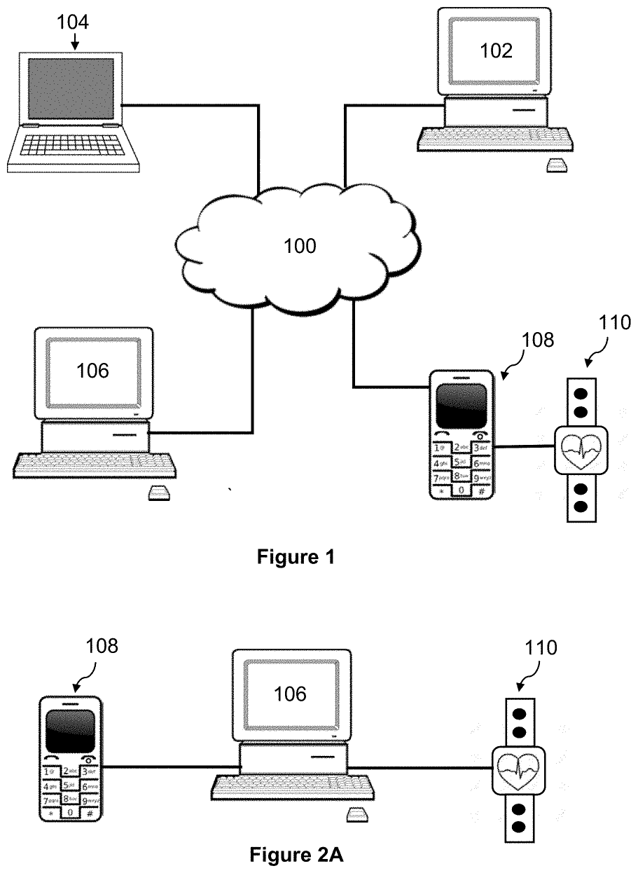

As shown in FIG. 1, a biometric device 110 may be in communication with a computing device 108, such as a smart phone, which, in turn, is in communication with at least one computing device (102, 104, 106) via a wide area network (“WAN”) 100, such as the Internet. The computing devices can be of different types, such as a PC, laptop, tablet, smart phone, smart watch etc., using one or different operating systems or platforms. In one embodiment of the present invention, the biometric device 110 is configured to acquire (e.g., measure, sense, estimate, etc.) an individual's heart rate (e.g., biometric data). The biometric data is then provided to the computing device 108, which includes a video and/or audio recorder (not shown).

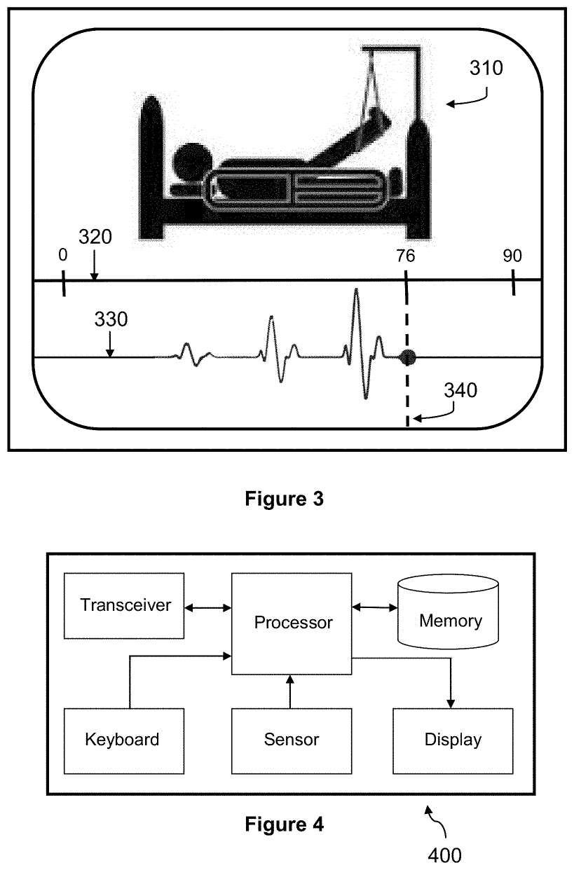

In a first embodiment of the present invention, the video and/or audio data are provided along with the heart rate data to a host computing device 106 via the network 100. Because the concurrent video and/or audio data and the heart rate data are provided to the host computing device 106, a host application operating thereon (not shown) can be used to synchronize the video data, audio data, and/or heart rate data, thereby allowing a user (e.g., via the user computing devices 102, 104) to view the video data and/or listen to the audio data (either in real-time or time delayed) while viewing the biometric data. For example, as shown in FIG. 3, the host application may use a time-stamp 320, or other sequencing method using metadata, to synchronize the video data 310 with the biometric data 330, allowing a user to view, for example, an individual (e.g., patient in a hospital, baby in a crib, etc.) at a particular time 340 (e.g., 76 seconds past the start time) and biometric data associated with the individual at that particular time 340 (e.g., 76 seconds past the start time).

It should be appreciated that the host application may further be configured to perform other functions, such as search for a particular activity in video data, audio data, biometric data and/or metadata, and/or ranking video data, audio data, and/or biometric data. For example, the host application may allow the user to search for a particular biometric event, such as a heart rate that has exceeded a particular threshold or value, a heart rate that has dropped below a particular threshold or value, a particular heart rate (or range) for a minimum period of time, etc. By way of another example, the host application may rank video data, audio data, biometric data, or a plurality of synchronized clips (e.g., highlight reels) chronologically, by biometric magnitude (highest to lowest, lowest to highest, etc.), by review (best to worst, worst to best, etc.), or by views (most to least, least to most, etc.). It should further be appreciated that such functions as the ranking, searching, and analysis of data is not limited to a user's individual session, but can be performed across any number of individual sessions of the user, as well as the session or number of sessions of multiple users. One use of this collection of all the various information (video, biometric and other) is to be able to generate sufficient data points for Big Data analysis and Machine Learning of the purposes of generating AI inferences and recommendations.

By way of example, machine learning algorithms could be used to search through video data automatically, looking for the most compelling content which would subsequently be stitched together into a short “highlight reel.” The neural network could be trained using a plurality of sports videos, along with ratings from users of their level of interest as the videos progress. The input nodes to the network could be a sample of change in intensity of pixels between frames along with the median excitement rating of the current frame. The machine learning algorithms could also be used, in conjunction with a multi-layer convolutional neural network, to automatically classify video content (e.g., what sport is in the video). Once the content is identified, either automatically or manually, algorithms can be used to compare the user's activity to an idealized activity. For example, the system could compare a video recording of the user's golf swing to that of a professional golfer. The system could then provide incremental tips to the user on how the user could improve their swing. Algorithms could also be used to predict fitness levels for users (e.g., if they maintain their program, giving them an incentive to continue working out), match users to other users or practitioners having similar fitness levels, and/or create routines optimized for each user.

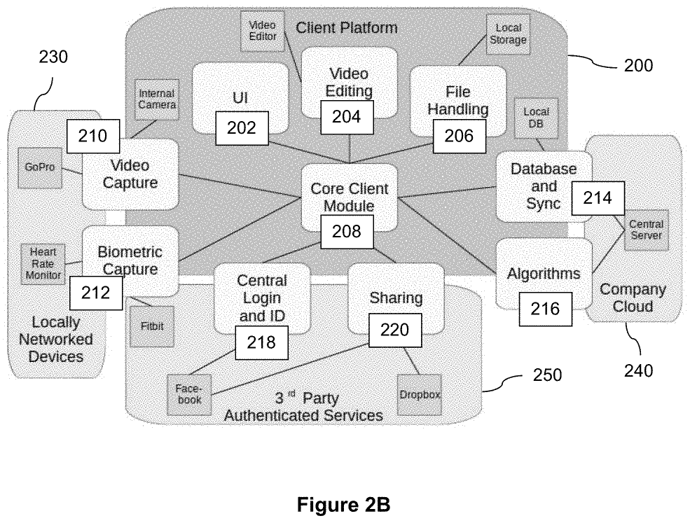

It should also be appreciated, as shown in FIG. 2A, that the biometric data may be provided to the host computing device 106 directly, without going through the computing device 108. For example, the computing device 108 and the biometric device 110 may communicate independently with the host computing device, either directly or via the network 100. It should further be appreciated that the video data, the audio data, and/or the biometric data need not be provided to the host computing device 106 in real-time. For example, video data could be provided at a later time as long as the data can be identified, or tied to a particular session. If the video data can be identified, it can then be synchronized to other data (e.g., biometric data) received in real-time.

In one embodiment of the present invention, as shown in FIG. 2B, the system includes a computing device 200, such as a smart phone, in communication with a plurality of devices, including a host computing device 240 via a WAN (see, e.g., FIG. 1 at 100), third party devices 250 via the WAN (see, e.g., FIG. 1 at 100), and local devices 230 (e.g., via wireless or wired connections). In a preferred embodiment, the computing device 200 downloads a program or application (i.e., client platform) from the host computing device 240 (e.g., company cloud). The client platform includes a plurality of modules that are configured to perform a plurality of functions.

For example, the client platform may include a video capture module 210 for receiving video data from an internal and/or external camera, and a biometric capture module 212 for receiving biometric data from an internal and/or external biometric device. The client platform may also include a user interface module 202, allowing a user to interact with the platform, a video editing module 204 for editing video data, a file handling module 206 for managing (e.g., storing, linking, etc.) data (e.g., video data, biometric data, identification data, start time data, duration data, sample rate data, self-realization data, time-stamp data, etc.), a database and sync module 214 for replicating data (e.g., copying data stored on the computing device 200 to the host computing device 240 and/or copying user data stored on the host computing device 240 to the computing device 200), an algorithm module 216 for processing received data (e.g., synchronizing data, searching/filtering data, creating a highlight reel, etc.), a sharing module 220 for sharing and/or storing data (e.g., video data, highlight reel, etc.) relating either to a single session or multiple sessions, and a central login and ID module 218 for interfacing with third party social media websites, such as Facebook™.

With respect to FIG. 2B, the computing device 200, which may be a smart phone, a tablet, or any other computing device, may be configured to download the client platform from the host computing device 240. Once the client platform is running on the computing device 200, the platform can be used to start a new session, receive video data for the session (i.e., via the video capture module 210) and receive biometric data for the session (i.e., via the biometric capture module 212). This data can be stored in local storage, in a local database, and/or on a remote storage device (e.g., in the company cloud or a third-party cloud, such as Dropbox™, etc.). In a preferred embodiment, the data is stored so that it is linked to information that (i) identifies the session and (ii) enables synchronization.

For example, video data is preferably linked to at least a start time (e.g., a start time of the session) and an identifier. The identifier may be a single number uniquely identifying the session, or a plurality of numbers (e.g., a plurality of globally (or universally) unique identifiers (GUIDs/UUIDs), where a first number uniquely identifying the session and a second number uniquely identifies an activity within the session, allowing a session (e.g., a trip to or an itinerary in a destination, such as Berlin) to include a plurality of activities (e.g., a bike ride, a walk, etc.). By way of example only, an activity (or session) identifier may be a 128 bit identifier that has a high probability of uniqueness, such as 8bf25512-f17a-4e9e-b49a-7c3f59ec1e85). The identifier may also include a session name and/or a session description. Other information about the video data (e.g., video length, video source, etc.) (i.e., “video metadata”) can also be stored and linked to the video data. Biometric data is preferably linked to at least the start time (e.g., the same start time linked to the video data), the identifier (e.g., the same identifier linked to the video data), and a sample rate, which identifies the rate at which biometric data is received and/or stored. For example, heart rate data may be received and stored at a rate of thirty samples per minute (30 spm), i.e., once every two seconds, or some other predetermined time interval sample.

In some cases, the sample rate used by the platform may be the sample rate of the biometric device (i.e., the rate at which data is provided by the biometric device). In other cases, the sample rate used by the platform may be independent from the rate at which data is received (e.g., a fixed rate, a configurable rate, etc.). For example, if the biometric device is configured to provide biometric data at a rate of sixty samples per minute (60 spm), the platform may still store the data at a rate of 30 spm. In other words, with a sample rate of 30 spm, the platform will have stored five values after ten seconds, the first value being the second value transmitted by the biometric device, the second value being the fourth value transmitted by the biometric device, and so on. Alternatively, if the biometric device is configured to provide biometric data only when the biometric data changes, the platform may still store the data at a rate of 30 spm. In this case, the first value stored by the platform may be the first value transmitted by the biometric device, the second value stored may be the first value transmitted by the biometric device if at the time of storage no new value has been transmitted by the biometric device, the third value stored may be the second value transmitted by the biometric device if at the time of storage a new value is being transmitted by the biometric device, and so on.

Once the video and biometric data is stored and linked, algorithms can be used to display the data together. For example, if biometric data is stored at a sample rate of 30 spm, which may be fixed or configurable, algorithms (e.g., 216) can be used to display a first biometric value (e.g., below the video data, superimposed over the video data, etc.) at the start of the video clip, a second biometric value two seconds later (two seconds into the video clip), a third biometric value two seconds later (four seconds into the video clip), etc. In alternate embodiments of the present invention, non-video data (e.g., biometric data, self-realization data, etc.) can be stored with a plurality of time-stamps (e.g., individual stamps or offsets for each stored value), which can be used together with the start time to synchronize non-video data to video data.

It should be appreciated that while the client platform can be configured to function autonomously (i.e., independent of the host network device 240), in one embodiment of the present invention, certain functions of the client platform are performed by the host network device 240, and can only be performed when the computing device 200 is in communication with the host computing device 240. Such an embodiment is advantageous in that it not only offloads certain functions to the host computing device 240, but it ensures that these functions can only be performed by the host computing device 240 (e.g., requiring a user to subscribe to a cloud service in order to perform certain functions). Functions offloaded to the cloud may include functions that are necessary to display non-video data together with video data (e.g., the linking of information to video data, the linking of information to non-video data, synchronizing non-video data to video data, etc.), or may include more advanced functions, such as generating and/or sharing a “highlight reel.” In alternate embodiments, the computing device 200 is configured to perform the foregoing functions as long as certain criteria has been met. This criteria may include the computing device 200 being in communication with the host computing device 240, or the computing device 200 previously being in communication with the host computing device 240 and the period of time since the last communication being equal to or less than a predetermined amount of time. Technology known to those skilled in the art (e.g., using a keyed hash-based method authentication code (HMAC), a stored time of said last communication (allowing said computing device to determine whether said delta is less than a predetermined amount of time), etc.) can be used to ensure that this criteria is met before allowing the performance of certain functions.

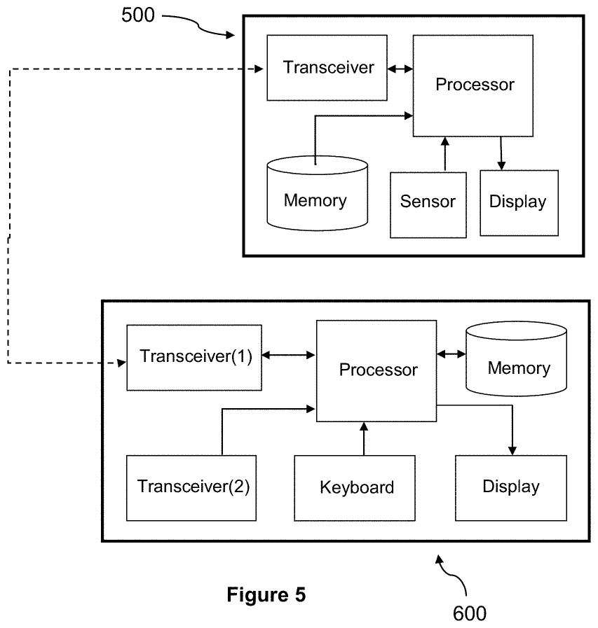

Block diagrams of an exemplary computing device and an exemplary biometric device are shown in FIG. 5. In particular, the exemplary biometric device 500 includes a sensor for sensing biometric data, a display for interfacing with the user and displaying various information (e.g., biometric data, set-up data, operation data, such as start, stop, and pause, etc.), a memory for storing the sensed biometric data, a transceiver for communicating with the exemplary computing device 600, and a processor for operating and/or driving the transceiver, memory, sensor, and display. The exemplary computing device 600 includes a transceiver(1) for receiving biometric data from the exemplary biometric device 500 (e.g., using any of telemetry, any WiFi standard, DNLA, Apple AirPlay, Bluetooth, near field communication (NFC), RFID, ZigBee, Z-Wave, Thread, Cellular, a wired connection, infrared or other method of data transmission, datacasting or streaming, etc.), a memory for storing the biometric data, a display for interfacing with the user and displaying various information (e.g., biometric data, set-up data, operation data, such as start, stop, and pause, input in-session comments or add voice notes, etc.), a keyboard for receiving user input data, a transceiver(2) for providing the biometric data to the host computing device via the Internet (e.g., using any of telemetry, any WiFi standard, DNLA, Apple AirPlay, Bluetooth, near field communication (NFC), RFID, ZigBee, Z-Wave, Thread, Cellular, a wired connection, infrared or other method of data transmission, datacasting or streaming, etc.), and a processor for operating and/or driving the transceiver(1), transceiver(2), keyboard, display, and memory.

The keyboard in the computing device 600, or alternatively the keyboard in biometric device 500, may be used to enter self-realization data, or data on how the user is feeling at a particular time. For example, if the user is feeling tired, the user may hit the “T” button on the keyboard. If the user is feeling their endorphins kick in, the user may hit the “E” button on the keyboard. And if the user is getting their second wind, the user may hit the “S” button on the keyboard. This data is then stored and linked to either a sample rate (like biometric data) or time-stamp data, which may be a time or an offset to the start time that each button was pressed. This would allow the self-realization data, in the same way as the biometric data, to be synchronized to the video data. It would also allow the self-realization data, like the biometric data, to be searched or filtered (e.g., in order to find video corresponding to a particular event, such as when the user started to feel tired, etc.).

It should be appreciated that the present invention is not limited to the block diagrams shown in FIG. 5, and a biometric device and/or a computing device that includes fewer or more components is within the spirit and scope of the present invention. For example, a biometric device that does not include a display, or includes a camera and/or microphone is within the spirit and scope of the present invention, as are other data-entry devices or methods beyond a keyboard, such as a touch screen, digital pen, voice/audible recognition device, gesture recognition device, so-called “wearable,” or any other recognition device generally known to those skilled in the art. Similarly, a computing device that only includes one transceiver, further includes a camera (for capturing video) and/or microphone (for capturing audio or for performing spatial analytics through recording or measurement of sound and how it travels), or further includes a sensor (see FIG. 4) is within the spirit and scope of the present invention. It should also be appreciated that self-realization data is not limited to how a user feels, but could also include an event that the user or the application desires to memorialize. For example, the user may want to record (or time-stamp) the user biking past wildlife, or a particular architectural structure, or the application may want to record (or time-stamp) a patient pressing a “request nurse” button, or any other sensed non-biometric activity of the user.

Referring back to FIG. 1, as discussed above in conjunction with FIG. 2B, the host application (or client platform) may operate on the computing device 108. In this embodiment, the computing device 108 (e.g., a smart phone) may be configured to receive biometric data from the biometric device 110 (either in real-time, or at a later stage, with a time-stamp corresponding to the occurrence of the biometric data), and to synchronize the biometric data with the video data and/or the audio data recorded by the computing device 108 (or a camera and/or microphone operating thereon). It should be appreciated that in this embodiment of the present invention, other than the host application being run locally (e.g., on the computing device 108), the host application (or client platform) operates as previously discussed.

Again, with reference to FIG. 1, in another embodiment of the present invention, the computing device 108 further includes a sensor for sensing biometric data. In this embodiment of the present invention, the host application (or client platform) operates as previously discussed (locally on the computing device 108), and functions to at least synchronize the video, audio, and/or biometric data, and allow the synchronized data to be played or presented to a user (e.g., via a display portion, via a display device connected directly to the computing device, via a user computing device connected to the computing device (e.g., directly, via the network, etc.), etc.).

It should be appreciated that the present invention, in any embodiment, is not limited to the computing devices (number or type) shown in FIGS. 1 and 2, and may include any of a computing, sensing, digital recording, GPS or otherwise location-enabled device (for example, using WiFi Positioning Systems “WPS”, or other forms of deriving geographical location, such as through network triangulation), generally known to those skilled in the art, such as a personal computer, a server, a laptop, a tablet, a smart phone, a cellular phone, a smart watch, an activity band, a heart-rate strap, a mattress sensor, a shoe sole sensor, a digital camera, a near field sensor or sensing device, etc. It should also be appreciated that the present invention is not limited to any particular biometric device, and includes biometric devices that are configured to be worn on the wrist (e.g., like a watch), worn on the skin (e.g., like a skin patch) or scalp, or incorporated into computing devices (e.g., smart phones, etc.), either integrated in, or added to items such as bedding, wearable devices such as clothing, footwear, helmets or hats, or ear phones, or athletic equipment such as rackets, golf clubs, or bicycles, where other kinds of data, including physical performance metrics such as racket or club head speed, or pedal rotation/second, or footwear recording such things as impact zones, gait or shear, can also be measured synchronously with biometrics, and synchronized to video. Other data can also be measured synchronously with video data, including biometrics on animals (e.g., a bull's acceleration or pivot or buck in a bull riding event, a horse's acceleration matched to heart rate in a horse race, etc.), and physical performance metrics of inanimate objects, such a revolutions/minute (e.g., in a vehicle, such as an automobile, a motorcycle, etc.), miles/hour (or the like) (e.g., in a vehicle, such as an automobile, a motorcycle, etc., a bicycle, etc.), or G-forces (e.g., experienced by the user, an animal, and inanimate object, etc.). All of this data (collectively “non-video data,” which may include metadata, or data on non-video data) can be synchronized to video data using a sample rate and/or at least one time-stamp, as discussed above.

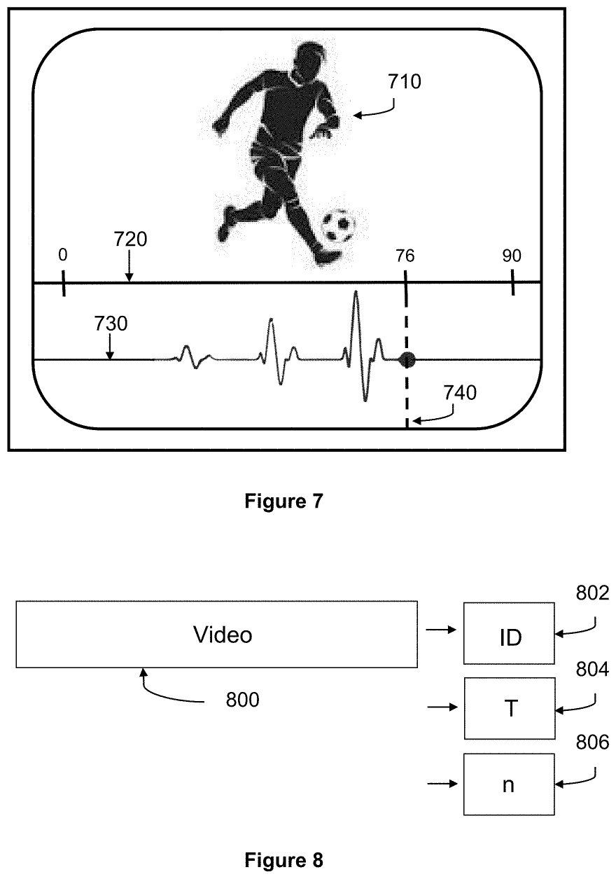

It should further be appreciated that the present invention need not operate in conjunction with a network, such as the Internet. For example, as shown in FIG. 2A, the biometric device 110, which may be, for example, be a wireless activity band for sensing heart rate, and the computing device 108, which may be, for example, a digital video recorder, may be connected directly to the host computing device 106 running the host application (not shown), where the host application functions as previously discussed. In this embodiment, the video, audio, and/or biometric data can be provided to the host application either (i) in real time, or (ii) at a later time, since the data is synchronized with a sample rate and/or time-stamp. This would allow, for example, at least video of an athlete, or a sportsman or woman (e.g., a football player, a soccer player, a racing driver, etc.) to be shown in action (e.g., playing football, playing soccer, motor racing, etc.) along with biometric data of the athlete in action (see, e.g., FIG. 7). By way of example only, this would allow a user to view a soccer player's heart rate 730 as the soccer player dribbles a ball, kicks the ball, heads the ball, etc. This can be accomplished using a time stamp 720 (e.g., start time, etc.), or other sequencing method using metadata (e.g., sample rate, etc.), to synchronize the video data 710 with the biometric data 730, allowing the user to view the soccer player at a particular time 740 (e.g., 76 seconds) and biometric data associated with the athlete at that particular time 340 (e.g., 76 seconds). Similar technology can be used to display biometric data on other athletes, card players, actors, online gamers, etc.

Where it is desirable to monitor or watch more than one individual from a camera view, for example, patients in a hospital ward being observed from a remote nursing station or, during a televised broadcast of a sporting event such as a football game, with multiple players on the sports field, the system can be so configured, by the subjects using Bluetooth or other wearable or NFC sensors (in some cases with their sensing capability also being location-enabled in order to identify which specific individual to track) capable of transmitting their biometrics over practicable distances, in conjunction with relays or beacons if necessary, such that the viewer can switch the selection of which of one or multiple individuals' biometric data to track, alongside the video or broadcast, and, if wanted and where possible within the limitations of the video capture field of the camera used, also to concentrate the view of the video camera on a reduced group or on a specific individual. In an alternate embodiment of the present invention, selection of biometric data is automatically accomplished, for example, based on the individual's location in the video frame (e.g., center of the frame), rate of movement (e.g., moving quicker than other individuals), or proximity to a sensor (e.g., being worn by the individual, embedded in the ball being carried by the individual, etc.), which may be previously activate or activated by a remote radio frequency signal. Activation of the sensor may result in biometric data of the individual being transmitted to a receiver, or may allow the receiver to identified biometric data of the individual amongst other data being transmitted (e.g., biometric data from other individuals).

In the context of fitness or sports tracking, it should be appreciated that the capturing of an individual's activity on video is not dependent on the presence of a third party to do this, but various methods of self-videoing can be envisaged, such as a video capture device mounted on the subject's wrist or a body harness, or on a selfie attachment or a gimbal, or fixed to an object (e.g., sports equipment such as bicycle handlebars, objects found in sporting environments such as a basketball or tennis net, a football goal post, a ceiling, etc., a drone-borne camera following the individual, a tripod, etc.). It should be further noted that such video capture devices can include more than one camera lens, such that not only the individual's activity may be videoed, but also simultaneously a different view, such as what the individual is watching or sees in front of them (i.e., the user's surroundings). The video capture device could also be fitted with a convex mirror lens, or have a convex mirror added as an attachment on the front of the lens, or be a full 360 degree camera, or multiple 360 cameras linked together, such that either with or without the use of specialized software known in the art, a 360 degree all-around or surround view can be generated, or a 360 global view in all axes can be generated.

In the context of augmented or virtual reality, where the individual is wearing suitably equipped augmented reality (“AR”) or virtual reality (“VR”) glasses, goggles, headset or is equipped with another type of viewing display capable of rendering AR, VR, or other synthesized or real 3D imagery, the biometric data such as heart rate from the sensor, together with other data such as, for example, work-out run or speed, from a suitably equipped sensor, such as an accelerometer capable of measuring motion and velocity, could be viewable by the individual, superimposed on their viewing field. Additionally an avatar of the individual in motion could be superimposed in front of the individual's viewing field, such that they could monitor or improve their exercise performance, or otherwise enhance the experience of the activity by viewing themselves or their own avatar, together (e.g., synchronized) with their performance (e.g., biometric data, etc.). Optionally, the biometric data also of their avatar, or the competing avatar, could be simultaneously displayed in the viewing field. In addition (or alternatively), at least one additional training or competing avatar can be superimposed on the individual's view, which may show the competing avatar(s) in relation to the individual (e.g., showing them superimposed in front of the individual, showing them superimposed to the side of the user, showing them behind the individual (e.g., in a rear-view-mirror portion of the display, etc.), and/or showing them in relation to the individual (e.g., as blips on a radar-screen portion of the display, etc.), etc. Competing avatar(s), either of real people such as their friends or training acquaintances, can be used to motivate the user to improve or correct their performance and/or to make their exercise routine more interesting (e.g., by allowing the individual to “compete” in the AR, VR, or Mixed Reality (“MR”) environment while exercising, or training, or virtually “gamifying” their activity through the visualization of virtual destinations or locations, imagined or real, such as historical sites, scanned or synthetically created through computer modeling).

Additionally, any multimedia sources to which the user is being exposed whilst engaging in the activity which is being tracked and recorded, should similarly be able to be recorded with the time stamp, for analysis and/or correlation of the individual's biometric response. An example of an application of this could be in the selection of specific music tracks for when someone is carrying out a training activity, where the correlation of the individual's past response, based, for example, on heart rate (and how well they achieved specific performance levels or objectives) to music type (e.g., the specific music track(s), a track(s) similar to the specific track(s), a track(s) recommended or selected by others who have listened to or liked the specific track(s), etc.) is used to develop a personalized algorithm, in order to optimize automated music selection to either enhance the physical effort, or to maximize recovery during and after exertion. The individual could further specify that they wished for the specific track or music type, based upon the personalized selection algorithm, to be played based upon their geographical location; an example of this would be someone who frequently or regularly uses a particular circuit for training or recreational purposes. Alternatively, tracks or types of music could be selected through recording or correlation of past biometric response in conjunction with self-realization inputting when particular tracks were being listened to.

It should be appreciated that biometric data does not need to be linked to physical movement or sporting activity, but may instead be combined with video of an individual at a fixed location (e.g., where the individual is being monitored remotely or recorded for subsequent review), for example, as shown in FIG. 3, for health reasons or a medical condition, such as in their home or in hospital, or a senior citizen in an assisted-living environment, or a sleeping infant being monitored by parents whilst in another room or location.

Alternatively, the individual might be driving past or in the proximity of a park or a shopping mall, with their location being recorded, typically by geo-stamping, or additional information being added by geo-tagging, such as the altitude or weather at the specific location, together with what the information or content is, being viewed or interacted with by the individual (e.g., a particular advertisement, a movie trailer, a dating profile, etc.) on the Internet or a smart/enabled television, or on any other networked device incorporating a screen, and their interaction with that information or content, being viewable or recorded by video, in conjunction with their biometric data, with all these sources of data being able to be synchronized for review, by virtue of each of these individual sources being time-stamped or the like (e.g., sampled, etc.). This would allow a third party (e.g., a service provider, an advertiser, a provider of advertisements, a movie production company/promoter, a poster of a dating profile, a dating site, etc.) to acquire for analysis of their response, the biometric data associated with the viewing of certain data by the viewer, where either the viewer or their profile could optionally be identifiable by the third party's system, or where only the identity of the viewer's interacting device is known, or can be acquired from the biometric sending party's GPS, or otherwise location-enabled, device.

For example, an advertiser or an advertisement provider could see how people are responding to an advertisement, or a movie production company/promoter could evaluate how people are responding to a movie trailer, or a poster of a dating profile or the dating site itself, could see how people are responding to the dating profile. Alternatively, viewers of online players of an online gaming or eSports broadcast service such as twitch.tv, or of a televised or streamed online poker game, could view the active participants' biometric data simultaneously with the primary video source as well as the participants' visible reactions or performance. As with video/audio, this can either be synchronized in real-time, or synchronized later using the embedded time-stamp or the like (e.g., sample rate, etc.). Additionally, where facial expression analysis is being generated from the source video, for example in the context of measuring an individual's response to advertising messages, since the video is already time-stamped (e.g., with a start time), the facial expression data can be synchronized and correlated to the physical biometric data of the individual, which has similarly been time-stamped and/or sampled,

As previously discussed, the host application may be configured to perform a plurality of functions. For example, the host application may be configured to synchronize video and/or audio data with biometric data. This would allow, for example, an individual watching a sporting event (e.g., on a TV, computer screen, etc.) to watch how each player's biometric data changes during play of the sporting event, or also to map those biometric data changes to other players or other comparison models. Similarly, a doctor, nurse, or medical technician could record a person's sleep habits, and watch, search or later review, the recording (e.g., on a TV, computer screen, etc.) while monitoring the person's biometric data. The system could also use machine learning to build a profile for each patient, identifying certain characteristics of the patient (e.g., their heart rate rhythm, their breathing pattern, etc.) and notify a doctor, a nurse, or medical technician or trigger an alarm if the measured characteristics appear abnormal or irregular.

The host application could also be configured to provide biometric data to a remote user via a network, such as the Internet. For example, a biometric device (e.g., a smart phone with a blood-alcohol sensor) could be used to measure a person's blood-alcohol level (e.g., while the person is talking to the remote user via the smart phone), and to provide the person's blood-alcohol level to the remote user. By placing the sensor near, or incorporating it in the microphone, such a system would allow a parent to determine whether their child has been drinking alcohol by participating in a telephone or video call with their child. Different sensors known in the art could be used to sense different chemicals in the person's breath, or detect people's breathing patterns through analysis of sound and speed variations, allowing the monitoring party to determine whether the subject has been using alcohol or other controlled substances or to conduct breath analysis for other diagnostic reasons.

The system could also be adapted with a so-called “lab on a chip” (LOC) integrated in the device itself, or with a suitable attachment added to it, for the remote testing for example, of blood samples where the smart-phone is either used for the collection and sending of the sample to a testing laboratory for analysis, or is used to carry out the sample collection and analysis within the device itself. In either case the system is adapted such that the identity of the subject and their blood sample are cross-authenticated for the purposes of sample and analysis integrity as well as patient identity certainty, through the simultaneous recording of the time-stamped video and time and/or location (or GPS) stamping of the sample at the point of collection and/or submission of the sample. This confirmation of identity is particularly important for regulatory, record keeping and health insurance reasons in the context of telemedicine, since the individual will increasingly be performing functions which, till now, have been carried out typically on-site at the relevant facility, by qualified and regulated medical or laboratory staff, rather than by the subject using a networked device, either for upload to the central analysis facility, or for remote analysis on the device itself.

This, or the collection of other biometric data such as heart rate or blood pressure, could also be applied in situations where it is critical for safety reasons, to check, via regular remote video monitoring in real time, whether say a pilot of a plane, a truck or train driver, are in fit and sound condition to be in control of their vehicle or vessel or whether for example they are experiencing a sudden incapacity or heart attack etc. Because the monitored person is being videoed at the same time as providing time-stamped, geo-stamped and/or sampled biometric data, there is less possibility for the monitored person or a third party, to “trick”, “spoof” or bypass the system. In a patient/doctor remote consultation setting, the system could be used for secure video consults where also, from a regulatory or health insurance perspective, the consultation and its occurrence is validated through the time and/or geo stamp validation. Furthermore, where there is a requirement for a higher level of authentication, the system could further be adapted to use facial recognition or biometric algorithms, to ensure that the correct person is being monitored, or facial expression analysis could be used for behavioral pattern assessment.

The concern that a monitored party would not wish to be permanently monitored (e.g., a senior citizen not wanting to have their every move and action continuously videoed) could be mitigated by the incorporation of various additional features. In one embodiment, the video would be permanently recording in a loop system which uses a reserved memory space, recording for a predetermined time period, and then, automatically erasing the video, where n represents the selected minutes in the loop and E is the event which prevents the recorded loop of n minutes being erased, and triggers both the real time transmission of the visible state or actions of the monitored person to the monitoring party, as well as the ability to rewind, in order for the monitoring party to be able to review the physical manifestation leading up to E. The trigger mechanism for E could be, for example, the occurrence of biometric data outside the predefined range, or the notification of another anomaly such as a fall alert, activated by movement or location sensors such as a gyroscope, accelerometer or magnetometer within the health band device worn by, say the senior citizen, or on their mobile phone or other networked motion-sensing device in their proximity. The monitoring party would be able not only to view the physical state of the monitored party after E, whilst getting a simultaneous read-out of their relevant biometric data, but also to review the events and biometric data immediately leading up to the event trigger notification. Alternatively, it could be further calibrated so that although video is recorded, as before, in the n loop, no video from the n loop will actually be transmitted to a monitoring party until the occurrence of E. The advantages of this system include the respect of the privacy of the individual, where only the critical event and the time preceding the event would be available to a third party, resulting also in a desired optimization of both the necessary transmission bandwidth and the data storage requirements. It should be appreciated that the foregoing system could also be configured such that the E notification for remote senior, infant or patient monitoring is further adapted to include facial tracking and/or expression recognition features.

Privacy could be further improved for the user if their video data and biometric data are stored by themselves, on their own device, or on their own external, or own secure third-party “cloud” storage, but with the index metadata of the source material, which enables the sequencing, extrapolation, searching and general processing of the source data, remaining at a central server, such as, in the case of medical records for example, at a doctor's office or other healthcare facility. Such a system would enable the monitoring party to have access to the video and other data at the time of consultation, but with the video etc. remaining in the possession of the subject. A further advantage of separating the hosting of the storage of the video and biometric source data from the treatment of the data, beyond enhancing the user's privacy and their data security, is that by virtue of its storage locally with the subject, not having to upload it to the computational server results both in reduced cost and increased efficiency of storage and data bandwidth. This would be of benefit also where such kind of remote upload of tests for review by qualified medical staff at a different location from the subject are occurring in areas of lower-bandwidth network coverage. A choice can also be made to lower the frame rate of the video material, provided that this is made consistent with sampling rate to confirm the correct time stamp, as previously described.

It should be appreciated that with information being stored at the central server (or the host device), various techniques known in the art can be implemented to secure the information, and prevent unauthorized individuals or entities from accessing the information. Thus, for example, a user may be provided (or allowed to create) a user name, password, and/or any other identifying (or authenticating) information (e.g., a user biometric, a key fob, etc.), and the host device may be configured to use the identifying (or authenticating) information to grant access to the information (or a portion thereof). Similar security procedures can be implemented for third parties, such as medical providers, insurance companies, etc., to ensure that the information is only accessible by authorized individuals or entities. In certain embodiments, the authentication may allow access to all the stored data, or to only a portion of the stored data (e.g., a user authentication may allow access to personal information as well as stored video and/or biometric data, whereas a third party authentication may only allow access to stored video and/or biometric data). In other embodiments, the authentication is used to determine what services are available to an individual or entity logging into the host device, or the website. For example, visitors to the website (or non-subscribers) may only be able to synchronize video/audio data to biometric data and/or perform rudimentary searching or other processing, whereas a subscriber may be able to synchronize video/audio data to biometric data and/or perform more detailed searching or other processing (e.g., to create a highlight reel, etc.).

It should further be appreciated that while there are advantages to keeping just the index metadata at the central server in the interests of storage and data upload efficiency as well as so providing a common platform for the interoperability of the different data types and storing the video and/or audio data on the user's own device (e.g., iCloud™, DropBox™, OneDrive™, etc.), the present invention is not so limited. Thus, in certain embodiments, where feasible, it may be beneficial to (1) store data (e.g., video, audio, biometric data, and metadata) on the user's device (e.g., allowing the user device to operate independent of the host device), (2) store data (e.g., video, audio, biometric data, and metadata) on the central server (e.g., host device) (e.g., allowing the user to access the data from any network-enabled device), or (3) store a first portion (e.g., video and audio data) on the user's device and store a second portion (e.g., biometric data and metadata) on the central server (e.g., host device) (e.g., allowing the user to only view the synchronized video/audio/biometric data when the user device is in communication with the host device, allowing the user to only search the biometric data (e.g., to create a “highlight reel”) or rank the biometric data (to identify and/or list data chronologically, magnitude (highest to lowest), magnitude (lowest to highest), best reviewed, worst reviewed, most viewed, least viewed, etc.) when the user device is in communication with the host device, etc.).

In another embodiment of the present invention, the functionality of the system is further (or alternatively) limited by the software operating on the user device and/or the host device. For example, the software operating on the user device may allow the user to play the video and/or audio data, but not to synchronize the video and/or audio data to the biometric data. This may be because the central server is used to store data critical to synchronization (time-stamp index, metadata, biometric data, sample rate, etc.) and/or software operating on the host device is necessary for synchronization. By way of another example, the software operating on the user device may allow the user to play the video and/or audio data, either alone or synchronized with the biometric data, but may not allow the user device (or may limit the user device's ability) to search or otherwise extrapolate from, or process the biometric data to identify relevant portions (e.g., which may be used to create a “highlight reel” of the synchronized video/audio/biometric data) or to rank the biometric and/or video data. This may be because the central server is used to store data critical to search and/or rank the biometric data (biometric data, biometric metadata, etc.), and/or software necessary for searching (or performing advanced searching of) and/or ranking (or performing advanced ranking of) the biometric data.

In any or all of the above embodiments, the system could be further adapted to include password or other forms of authentication to enable secured access (or deny unauthorized access) to the data in either of one or both directions, such that the user requires permission to access the host, or the host to access the user's data. Where interaction between the user and the monitoring party or host is occurring in real time such as in a secure video consult between patient and their medical practitioner or other medical staff, data could be exchanged and viewed through the establishment of a Virtual Private Network (VPN). The actual data (biometric, video, metadata index, etc.) can alternatively or further be encrypted both at the data source, for example at the individual's storage, whether local or cloud-based, and/or at the monitoring reviewing party, for example at patient records at the medical facility, or at the host administration level.

In the context of very young infant monitoring, a critical and often unexplained problem is Sudden Infant Death Syndrome (SIDS). Whilst the incidences of SIDS are often unexplained, various devices attempt to prevent its occurrence. However, by combining the elements of the current system to include sensor devices in or near the baby's crib to measure relevant biometric data including heart rate, sleep pattern, breath analyzer, and other measures such as ambient temperature, together with a recording device to capture movement, audible breathing, or lack thereof (i.e., silence) over a predefined period of time, the various parameters could be set in conjunction with the time-stamped video record, by the parent or other monitoring party, to provide a more comprehensive alert, to initiate a more timely action or intervention by the user, or indeed to decide that no action response would in fact be necessary. Additionally, in the case, for example, of a crib monitoring situation, the system could be so configured to develop from previous observation, with or without input from a monitoring party, a learning algorithm to help in discerning what is “normal,” what is false positive, or what might constitute an anomaly, and therefore a call to action.

The host application could also be configured to play video data that has been synchronized to biometric data, or search for the existence of certain biometric data. For example, as previously discussed, by video recording with sound a person sleeping, and synchronizing the recording with biometric data (e.g., sleep patterns, brain activity, snoring, breathing patterns, etc.), the biometric data can be searched to identify where certain measures such as sound levels, as measured for example in decibels, or periods of silences, exceed or drop below a threshold value, allowing the doctor, nurse, or medical technician to view the corresponding video portion without having to watch the entire video of the person sleeping.

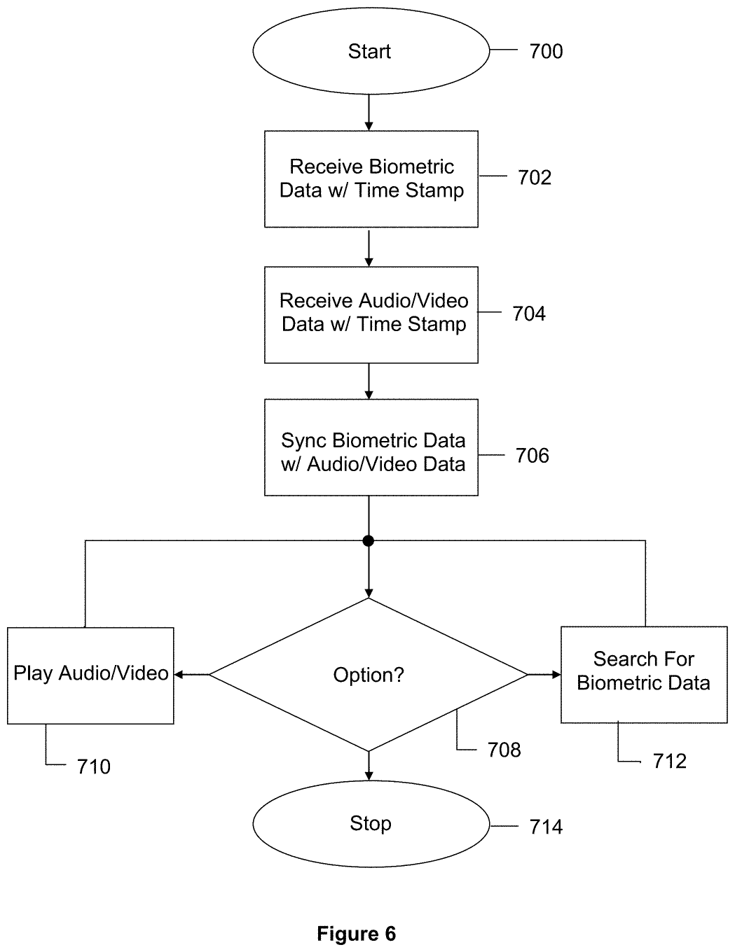

Such a method is shown in FIG. 6, starting at step 700, where biometric data and time stamp data (e.g., start time, sample rate) is received (or linked) at step 702. Audio/video data and time stamp data (e.g., start time, etc.) is then received (or linked) at step 704. The time stamp data (from steps 702 and 704) is then used to synchronize the biometric data with the audio/video data. The user is then allowed to operate the audio/video at step 708. If the user selects play, then the audio/video is played at step 710. If the user selects search, then the user is allowed to search the biometric data at step 712. Finally, if the user selects stop, then the video is stopped at step 714.

It should be appreciated that the present invention is not limited to the steps shown in FIG. 6. For example, a method that allows a user to search for biometric data that meets at least one condition, play the corresponding portion of the video (or a portion just before the condition), and stop the video from playing after the biometric data no longer meets the at least one condition (or just after the biometric data non longer meets the condition) is within the spirit and scope of the present invention. By way of another example, if the method involves interacting between the user device and the host device to synchronize the video/audio data and the biometric data and/or search the biometric data, then the method may further involve the steps of uploading the biometric data and/or metadata to the host device (e.g., in this embodiment the video/audio data may be stored on the user device), and using the biometric data and/or metadata to create a time-stamp index for synchronization and/or to search the biometric data for relevant or meaningful data (e.g., data that exceeds a threshold, etc.). By way of yet another example, the method may not require step 706 if the audio/video data and the biometric data are played together (synchronized) in real-time, or at the time the data is being played (e.g., at step 710).

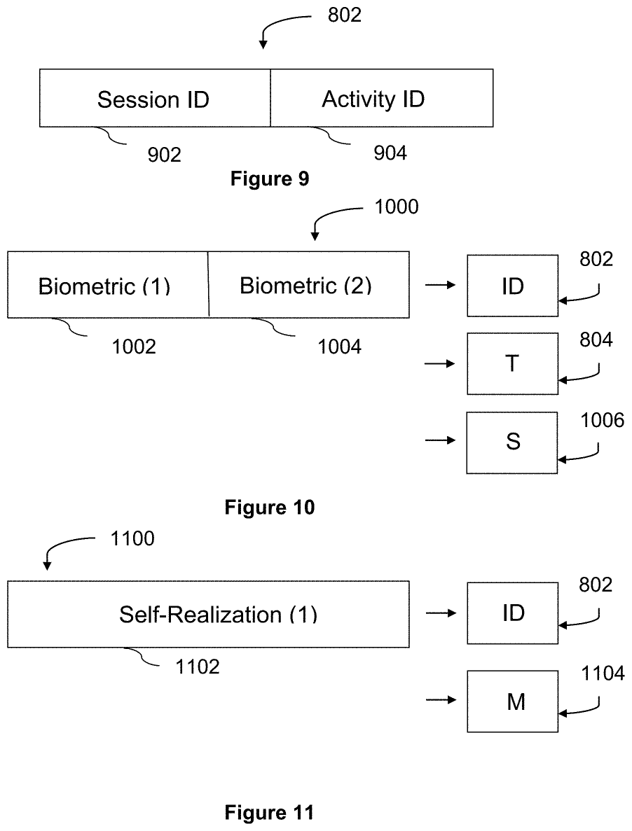

In one embodiment of the present invention, as shown in FIG. 8, the video data 800, which may also include audio data, starts at a time “T” and continues for a duration of “n.” The video data is preferably stored in memory (locally and/or remotely) and linked to other data, such as an identifier 802, start time 804, and duration 806. Such data ties the video data to at least a particular session, a particular start time, and identifies the duration of the video included therein. In one embodiment of the present invention, each session can include different activities. For example, a trip to a destination in Berlin, or following a specific itinerary on a particular day (session) may involve a bike ride through the city (first activity) and a walk through a park (second activity). Thus, as shown in FIG. 9, the identifier 802 may include both a session identifier 902, uniquely identifying the session via a globally unique identifier (GUID), and an activity identifier 904, uniquely identifying the activity via a globally unique identifier (GUID), where the session/activity relationship is that of a parent/child.

In one embodiment of the present invention, as shown in FIG. 10, the biometric data 1000 is stored in memory and linked to the identifier 802 and a sample rate “m” 1104. This allows the biometric data to be linked to video data upon playback. For example, if identifier 802 is one, start time 804 is 1:00 PM, video duration is one minute, and the sample rate 1104 is 30 spm, then the playing of the video at 2:00 PM would result in the first biometric value (biometric (1)) to be displayed (e.g., below the video, over the video, etc.) at 2:00 PM, the second biometric value (biometric (2)) to be displayed (e.g., below the video, over the video, etc.) two seconds later, and so on until the video ends at 2:01 PM. While self-realization data can be stored like biometric data (e.g., linked to a sample rate), if such data is only received periodically, it may be more advantageous to store this data 110 as shown in FIG. 11, i.e., linked to the identifier 802 and a time-stamp 1104, where “m” is either the time that the self-realization data 1100 was received or an offset between this time and the start time 804 (e.g., ten minutes and four seconds after the start time, etc.).

This can be seen, for example, in FIG. 14, where video data starts at time T, biometric data is sampled every two seconds (30 spm), and self-realization data is received at time T+3 (or three units past the start time). While the video 1402 is playing, a first biometric value 1404 is displayed at time T+1, first self-realization data 1406 is displayed at time T+2, and a second biometric value 1406 is displayed at time T+4. By storing data in this fashion, both video and non-video data can be stored separately from one another and synchronized in real-time, or at the time the video is being played. It should be appreciated that while separate storage of data may be advantageous for devices having minimal memory and/or processing power, the client platform may be configured to create new video data, or data that includes both video and non-video data displayed synchronously. Such a feature may advantageous in creating a highlight reel, which can then be shared using social media websites, such as Facebook™ or Youtube™, and played using standard playback software, such as Quicktime™. As discussed in greater detail below, a highlight reel may include various portions (or clips) of video data (e.g., when certain activity takes place, etc.) along with corresponding biometric data.

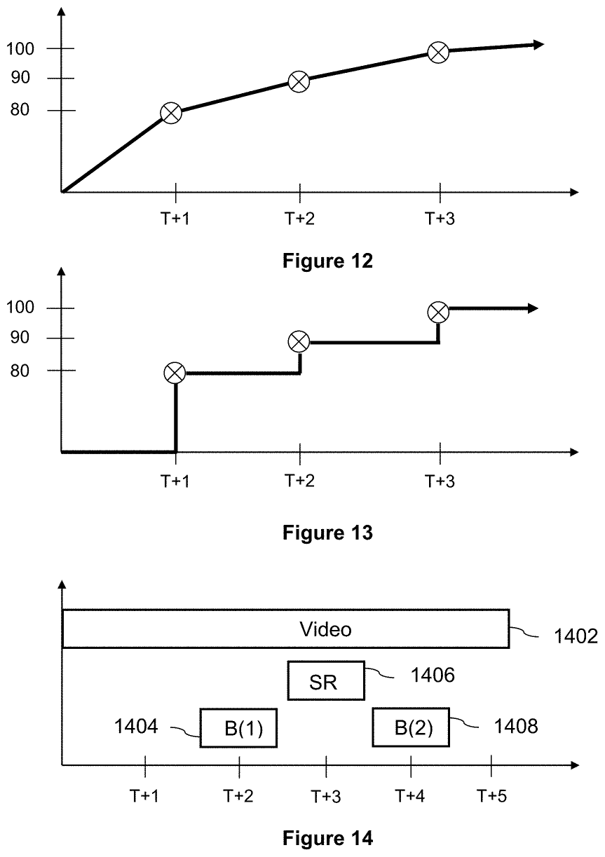

When sampled data is subsequently displayed, the client platform can be configured to display this data using certain extrapolation techniques. For example, in one embodiment of the present invention, as shown in FIG. 12, where a first biometric value 1202 is displayed at T+1, a second biometric value 1204 is displayed at T+2, and a third biometric value 1206 is displayed at T+3, biometric data can be displayed at non-sampled times using known extrapolation techniques, including linear and non-linear interpolation and all other extrapolation and/or interpolation techniques generally known to those skilled in the art. In another embodiment of the present invention, as shown in FIG. 13, the first biometric value 1202 remains on the display until the second biometric value 1204 is displayed, the second biometric value 1204 remains on the display until the third biometric value 1206 is displayed, and so on.

With respect to linking data to an identifier, which may be linked to other data (e.g., start time, sample rate, etc.), if the data is received in real-time, the data can be linked to the identifier(s) for the current session (and/or activity). However, when data is received after the fact (e.g., after a session has ended), there are several ways in which the data can be linked to a particular session and/or activity (or identifier(s) associated therewith). The data can be manually linked (e.g., by the user) or automatically linked via the application. With respect to the latter, this can be accomplished, for example, by comparing the duration of the received data (e.g., the video length) with the duration of the session and/or activity, by assuming that the received data is related to the most recent session and/or activity, or by analyzing data included within the received data. For example, in one embodiment, data included with the received data (e.g., metadata) may identify a time and/or location associated with the data, which can then be used to link the received data to the session and/or activity. In another embodiment, the computing device could display or play data (e.g., a barcode, such as a QR code, a sound, such as a repeating sequence of notes, etc.) that identifies the session and/or activity. An external video/audio recorder could record the identifying data (as displayed or played by the computing device) along with (e.g., before, after, or during) the user and/or his/her surroundings. The application could then search the video/audio data for identifying data, and use this data to link the video/audio data to a session and/or activity. The identifying portion of the video/audio data could then be deleted by the application if desired. In an alternate embodiment, a barcode (e.g., a QR code) could be printed on a physical device (e.g., a medical testing module, which may allow communication of medical data over a network (e.g., via a smart phone)) and used (as previously described) to synchronize video of the user using the device to data provided by the device. In the case of a medical testing module, the barcode printed on the module could be used to synchronize video of the testing to the test result provided by the module. In yet another embodiment, both the computing device and the external video/audio recorder are used to record video and/or audio of the user (e.g., the user stating “begin Berlin biking session,” etc.) and to use the user-provided data to link the video/audio data to a session and/or activity. For example, the computing device may be configured to link the user-provided data with a particular session and/or activity (e.g., one that is started, one that is about to start, one that just ended, etc.), and to use the user-provided data in the video/audio data to link the video/audio data to the particular session and/or activity.

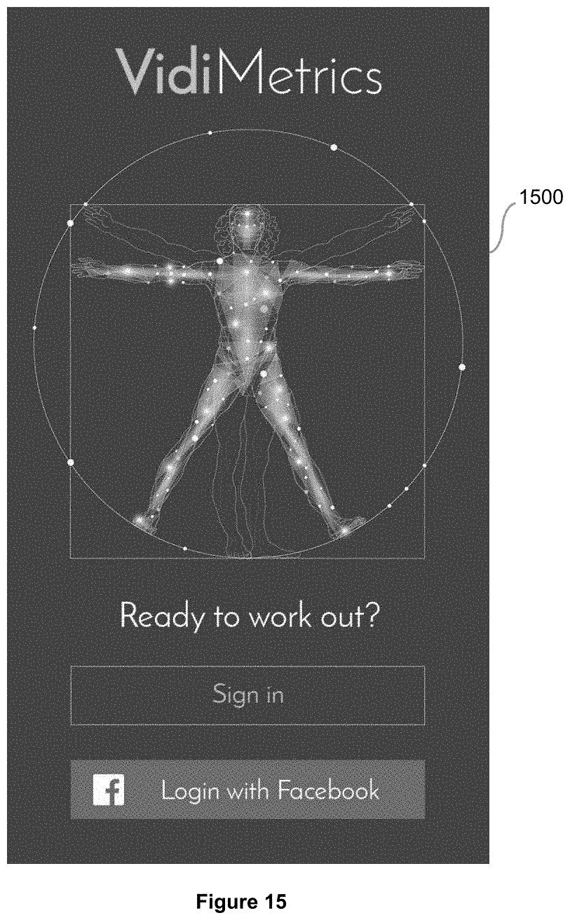

In one embodiment of the present invention, the client platform (or application) is configured to operate on a smart phone or a tablet. The platform (either alone or together with software operating on the host device) may be configured to create a session, receive video and non-video data during the session, and playback video data together (synchronized) with non-video data. The platform may also allow a user to search for a session, search for certain video and/or non-video events, and/or create a highlight reel. FIGS. 15-29 show exemplary screen shots of such a platform.

For example, FIG. 15 shows an exemplary “sign in” screen 1500, allowing a user to sign into the application and have access to application-related, user-specific data, as stored on the computing device and/or the host computing device. The login may involve a user ID and password unique to the application, the company cloud, or a social service website, such as Facebook™.

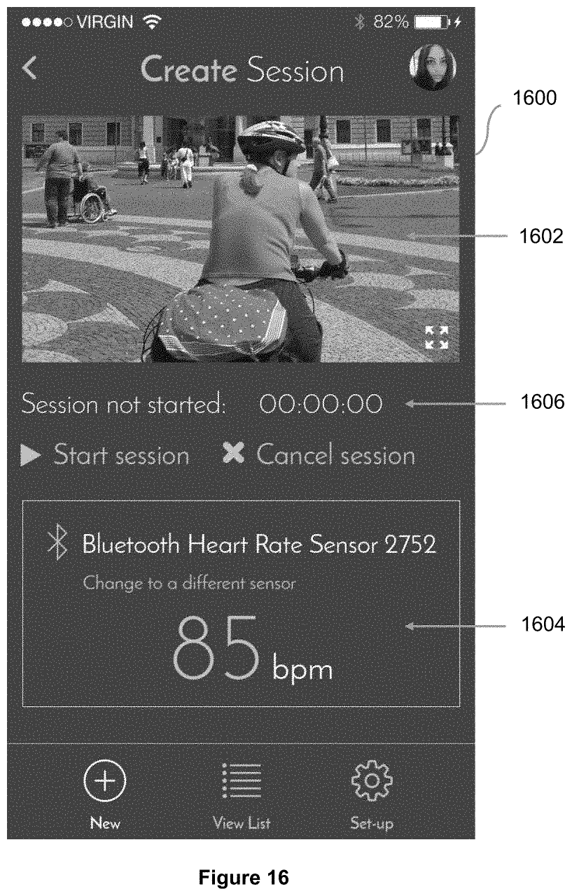

Once the user is signed in, the user may be allowed to create a session via an exemplary “create session” screen 1600, as shown in FIG. 16. In creating a session, the user may be allowed to select a camera (e.g., internal to the computing device, external to the computing device (e.g., accessible via the Internet, connected to the computing device via a wired or wireless connection), etc.) that will be providing video data. Once a camera is selected, video data 1602 from the camera may be displayed on the screen. The user may also be allowed to select a biometric device (e.g., internal to the computing device, external to the computing device (e.g., accessible via the Internet, connected to the computing device via a wired or wireless connection), etc.) that will be providing biometric data. Once a biometric device is selected, biometric data 1604 from the biometric device may be displayed on the screen. The user can then start the session by clicking the “start session” button 1608. While the selection process is preferably performed before the session is started, the user may defer selection of the camera and/or biometric device until after the session is over. This allows the application to receive data that is not available in real-time, or is being provided by a device that is not yet connected to the computing device (e.g., an external camera that will be plugged into the computing device once the session is over).

It should be appreciated that in a preferred embodiment of the present invention, clicking the “start session” button 1608 not only starts a timer 1606 that indicates a current length of the session, but it triggers a start time that is stored in memory and linked to a globally unique identifier (GUID) for the session. By linking the video and biometric data to the GUID, and linking the GUID to the start time, the video and biometric data is also (by definition) linked to the start time. Other data, such as sample rate, can also be linked to the biometric data, either by linking the data to the biometric data, or linking the data to the GUID, which is in turn linked to the biometric data.

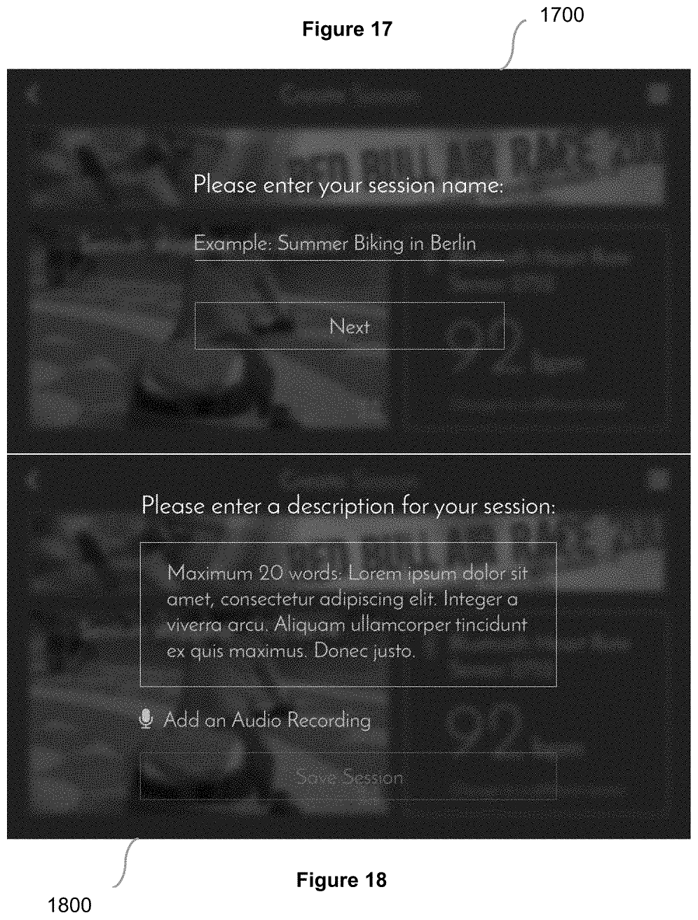

Either before the session is started, or after the session is over, the user may be allowed to enter a session name via an exemplary “session name” screen 1700, as shown in FIG. 17. Similarly, the user may also be allowed to enter a session description via an exemplary “session description” screen 1800, as shown in FIG. 18.

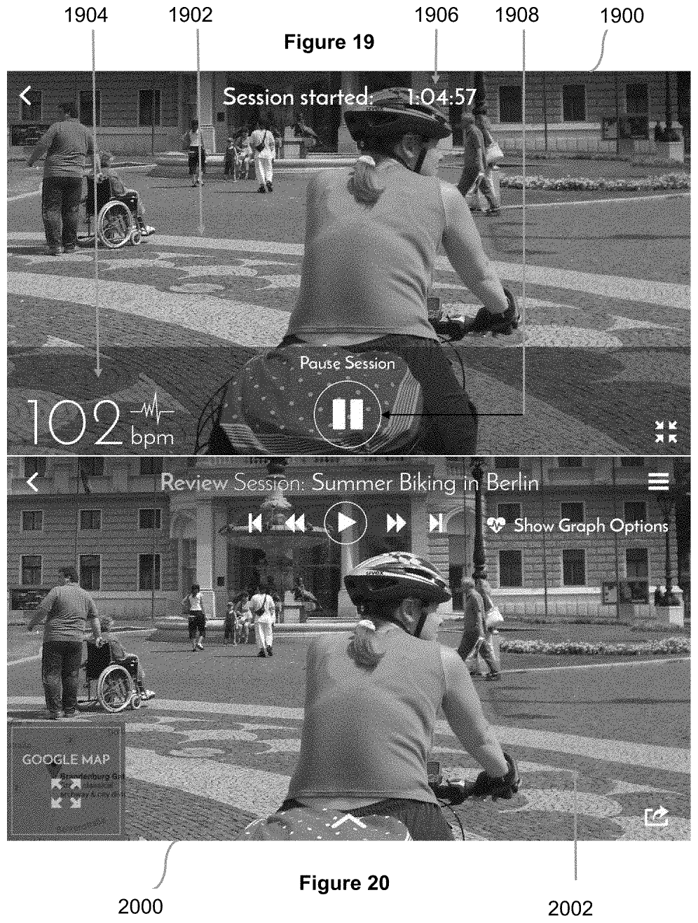

FIG. 19 shows an exemplary “session started” screen 1900, which is a screen that the user might see while the session is running. On this screen, the user may see the video data 1902 (if provided in real-time), the biometric data 1904 (if provided in real-time), and the current running time of the session 1906. If the user wishes to pause the session, the user can press the “pause session” button 1908, or if the user wishes to stop the session, the user can press the “stop session” button (not shown). By pressing the “stop session” button (not shown), the session is ended, and a stop time is stored in memory and linked to the session GUID. Alternatively, by pressing the “pause session” button 1908, a pause time (first pause time) is stored in memory and linked to the session GUID. Once paused, the session can then be resumed (e.g., by pressing the “resume session” button, not shown), which will result in a resume time (first resume time) to be stored in memory and linked to the session GUID. Regardless of whether a session is started and stopped (i.e., resulting in a single continuous video), or started, paused (any number of times), resumed (any number of times), and stopped (i.e., resulting in a plurality of video clips), for each start/pause time stored in memory, there should be a corresponding stop/resume time stored in memory.

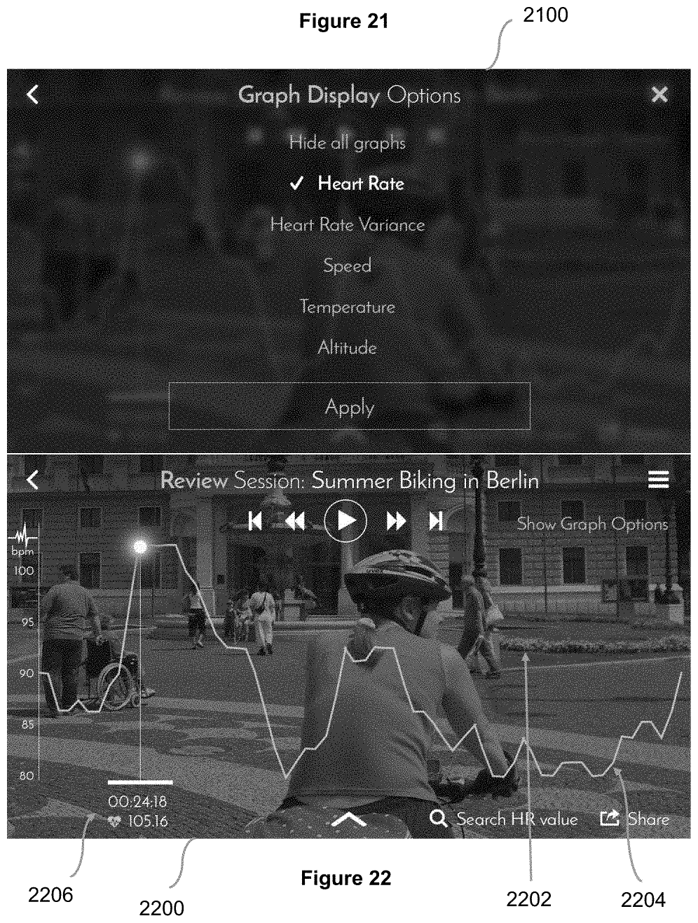

Once a session has been stopped, it can be reviewed via an exemplary “review session” screen 2000, as shown in FIG. 20. In its simplest form, the review screen may playback video data linked to the session (e.g., either a single continuous video if the session does not include at least one pause/resume, multiple video clips played one after another if the session includes at least one pause/resume, or multiple video clips played together if the multiple video clips are related to one another (e.g., two videos (e.g., from different vantage points) of the user performing a particular activity, a first video of the user performing a particular activity while viewing a second video, such as a training video). If the user wants to see non-video data displayed along with the video data, the user can press the “show graph options” button 2022. By pressing this button, the user is presented with an exemplary “graph display option” screen 2100, as shown in FIG. 21. Here, the user can select data that he/she would like to see along with the video data, such as biometric data (e.g., heart rate, heart rate variance, user speed, etc.), environmental data (e.g., temperature, altitude, GPS, etc.), or self-realization data (e.g., how the user felt during the session). FIG. 22 shows an exemplary “review session” screen 2000 that includes both video data 2202 and biometric data, which may be shown in graph form 2204 or written form 2206. If more than one individual can be seen in the video, the application may be configured to show biometric data on each individual, either at one time, or as selected by the user (e.g., allowing the user to view biometric data on a first individual by selecting the first individual, allowing the user to view biometric data on a second individual by selecting the second individual, etc.).

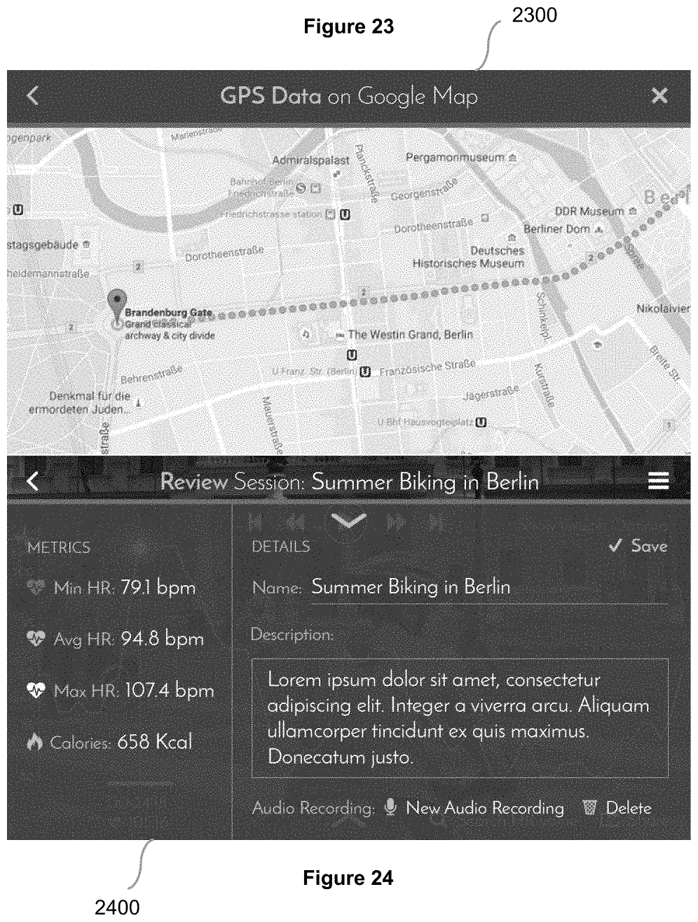

FIG. 23 shows an exemplary “map” screen 2300, which may be used to show GPS data to the user. Alternatively, GPS data can be presented together with the video data (e.g., below the video data, over the video data, etc.). An exemplary “summary” screen 2400 of the session may also be presented to the user (see FIG. 24), displaying session information such as session name, session description, various metrics, etc.

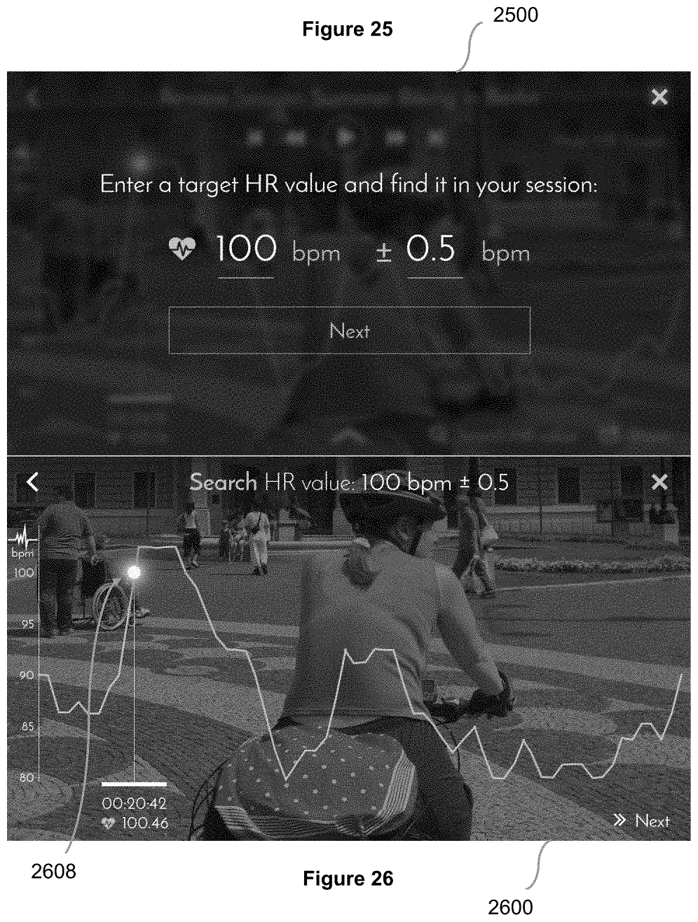

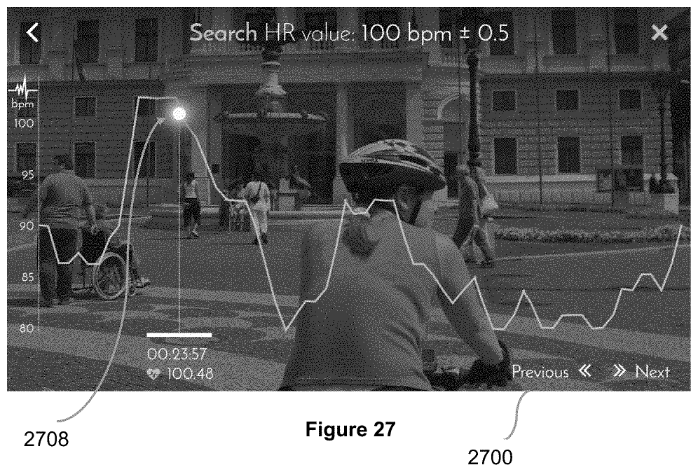

By storing video and non-video data separately, the data can easily be searched. For example, FIG. 25 shows an exemplary “biometric search” screen 2500, where a user can search for a particular biometric value or range (i.e., a biometric event). By way of example, the user may want to jump to a point in the session where their heart rate is between 95 and 105 beats-per-minute (bpm). FIG. 26 shows an exemplary “first result” screen 2600 where the user's heart rate is at 100.46 bmp twenty minutes and forty-two seconds into the session (see, e.g., 2608). FIG. 27 shows an exemplary “second result” screen 2700 where the user's heart rate is at 100.48 bmp twenty-three minutes and forty-eight seconds into the session (see, e.g., 2708). It should be appreciated that other events can be searched for in a session, including video events and self-realization events.

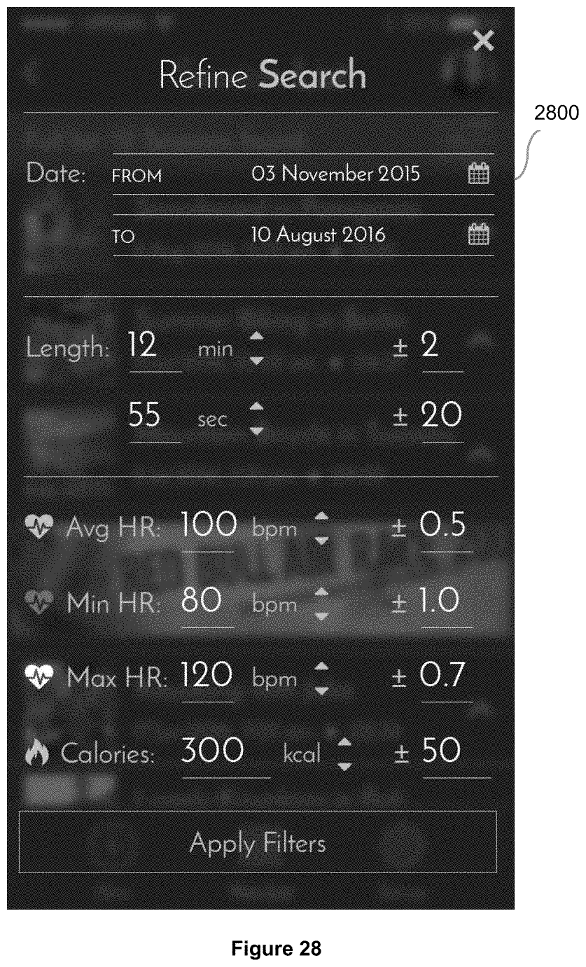

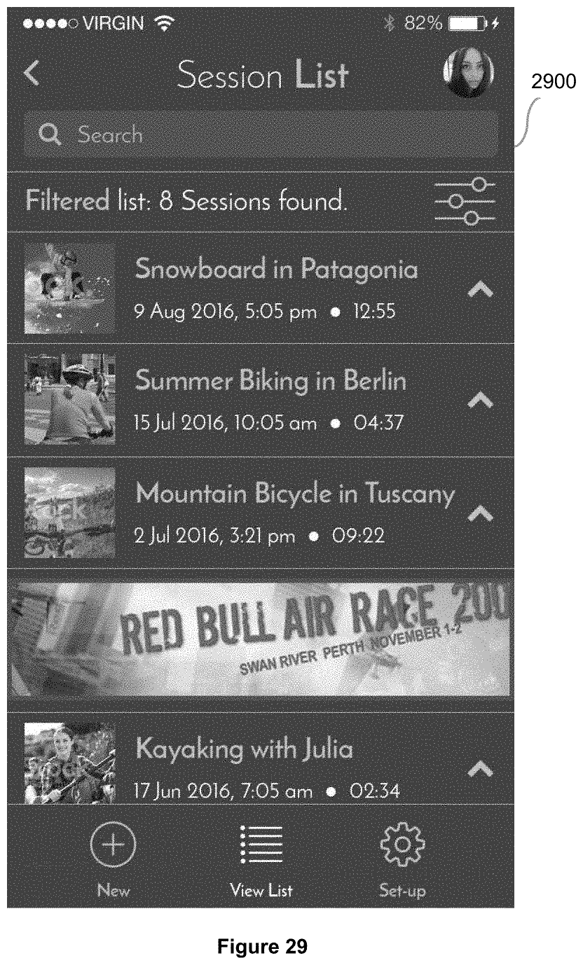

Not only can data within a session be searched, but so too can data from multiple sessions. For example, FIG. 28 shows an exemplary “session search” screen 2800, where a user can enter particular search criteria, including session date, session length, biometric events, video event, self-realization event, etc. FIG. 29 shows an exemplary “list” screen 2900, showing sessions that meet the entered criteria.

The present invention (in second part) is described as personalization preference optimization, or using at least one emotional state, mood, physical state, or mental state (“state”) of an individual (e.g., determined using biometric data from the individual, etc.) to determine a response, which may include web-based data that is provided to the individual as a result of the at least one state, either alone or together with other data (e.g., at least one thing (or data related thereto) in a proximity of the individual at a time that the individual is experiencing the at least one emotion, etc.).

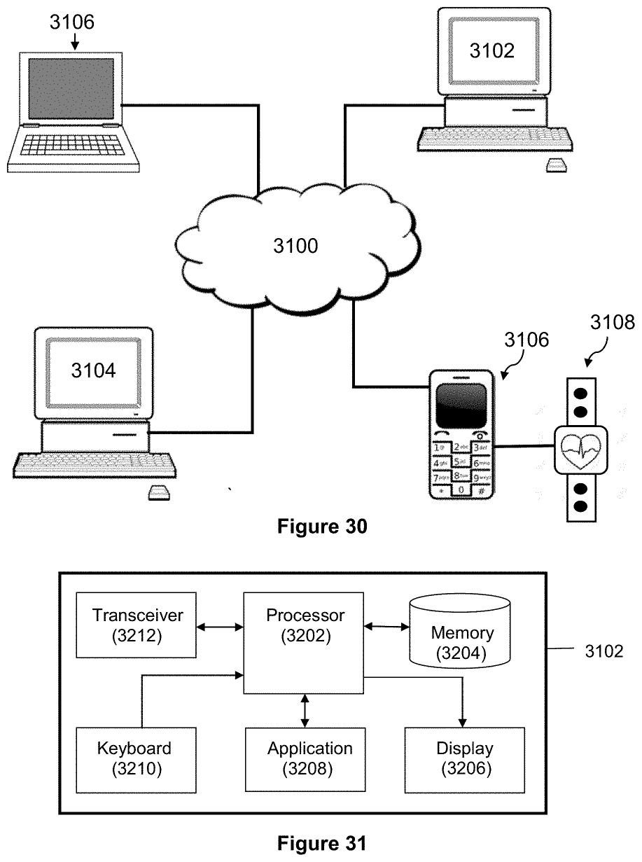

As shown in FIG. 30, preferred embodiments of the present invention operate in accordance with a Web host 3102 in communication with at least content provider (e.g., provider of web-based data) 3104 and at least one network device 3106 via a wide area network (WAN) 3100, wherein each network device 3106 is operated by an individual and is configured to communicate biometric data of the individual to the Web host 3102, where the biometric data is acquired using at least one biometric sensor 3108.

While FIG. 30 depicts the preferred embodiment, it should be appreciated that other embodiments are within the spirit and scope of the present invention. For example, the network device 3106 itself may be configured to collect (e.g., sense, etc.) biometric data on the individual. This may be accomplished, for example, through the use of at least one microphone (e.g., to acquire voice data from the individual), at least one camera (e.g., to acquire video data on the individual), at least one heart rate sensor (e.g., to measure heart rate data on the individual), at least one breath sensor (e.g., to measure breath chemical composition of the individual), etc. By way of another example, the host may be configured to communicate directly with the network device, for example using a wireless protocol such as Bluetooth, Wi-Fi, etc. By way of yet another example, the host may be configured to acquire biometric data directly from the individual using, for example, at least one microphone, at least one camera, or at least one sensor (e.g., a heart rate sensor, a breath sensor, etc.). In this example, the host may be configured to provide data to the individual (e.g., display data on a host display) or perform at least one action (e.g., switch an automobile to autopilot, restrict speed, etc.).

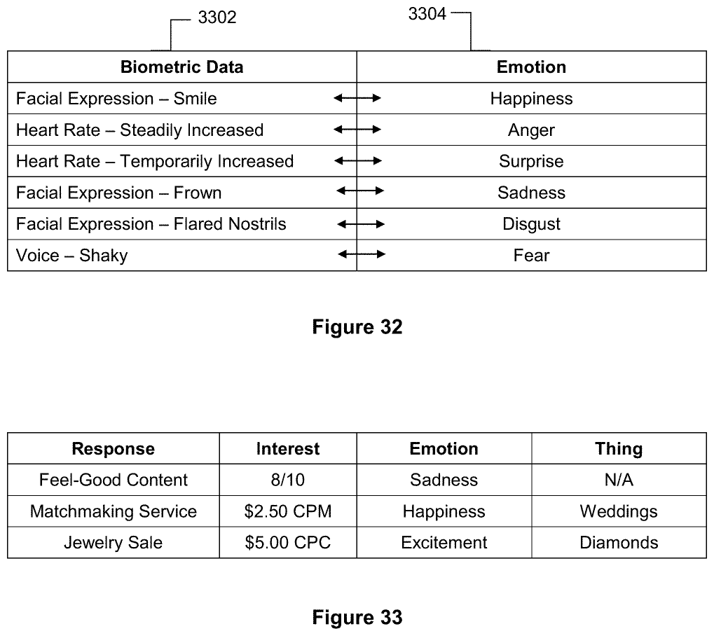

With reference to FIGS. 30 and 31, the content provider 3104 provides the Web host 3102 with web-based data, such as a website, a web page, image data, video data, audio data, an advertisement, etc. Other web-based data is further provided to the Web host 3102 by at least one other content provider (not shown). The plurality of web-based data (e.g., plurality of websites, plurality of web pages, plurality of image data, plurality of video data, plurality of audio data, plurality of advertisements, etc.) is stored in a memory device 3204 along with other data (discussed below), such as information that links different biometric data to different states (see FIG. 32) and interest data (see FIG. 33). It should be appreciated that the present invention is not limited to the memory device 3204 depicted in FIG. 31, and may include additional memory devices (e.g., databases, etc.), internal and/or external to the Web host 3102.

The Web host 3102 is then configured to receive biometric data from the network device 3106. As discussed above, the biometric data is preferably related to (i.e., acquired from) an individual who is operating the network device 3106, and may be received using at least one biometric sensor 3108, such as an external heart rate sensor, etc. As discussed above, the present invention is not limited to the biometric sensor 3108 depicted in FIG. 30, and may include additional (or different) biometric sensors (or the like, such as microphones, cameras, etc.) that are external to the network device 3106, and/or at least one biometric sensor (or the like, such as microphones, cameras, etc.) internal to the network device. If the biometric sensor is external to the network device, it may communicate with the network device via at least one wire and/or wirelessly (e.g., Bluetooth, Wi-Fi, etc.).

It should be appreciated that the present invention is not limited to any particular type of biometric data, and may include, for example, heart rate, blood pressure, breathing rate, temperature, eye dilation, eye movement, facial expressions, speech pitch, auditory changes, body movement, posture, blood hormonal levels, urine chemical concentrations, breath chemical composition, saliva chemical composition, and/or any other types of measurable physical or biological characteristics of the individual. The biometric data may be a particular value (e.g., a particular heart rate, etc.) or a change in value (e.g., a change in heart rate), and may be related to more than one characteristic (e.g., heart rate and breathing rate).

It should also be appreciated that while best results come from direct measurement of known individuals, the same methods of correlation can be applied to general categories of people. An example is that a facial recognition system may know that 90% of the people at a particular location, such as a hospital, are fearful and that an individual is known to be at that location. Even if biometric data of that individual is not shared with the system, the correlation may be applied, preserving privacy and still allowing for statistically significant targeting. Another example would be a bar that had urine chemical analyzers integrated into the bathrooms, providing general information about people at the bar. This data could then be coordinated with time and location back to a group of people and provide significant correlations for targeting messages to an individual (e.g., an individual who was at the bar during that time).

As shown in FIG. 31, the Web host 3102 includes an application 3208 that is configured to determine at least one state from the received biometric data. This is done using known algorithms and/or correlations between biometric data and different states, such as emotional states, as stored in the memory device 3204. For example, as shown in FIG. 32, if the biometric data 3302 indicates that the individual is smiling (e.g., via use of at least one camera), then it may be determined that the individual is experiencing the emotion 3304 of happiness. By way of other examples, if the biometric data 3302 indicates that the individual's heart rate is steadily increasing (e.g., via use of a heart rate sensor), then it may be determined that the individual is experiencing the emotion 3304 of anger. If the biometric data 3302 indicates that the individual's heart rate temporarily increases (e.g., via use of a heart rate sensor), then it may be determined that the individual is experiencing the emotion 3304 of surprise. If the biometric data 3302 indicates that the individual is frowning (e.g., via use of at least one camera), then it may be determined that the individual is experiencing the emotion 3304 of sadness. If the biometric data 3302 indicates that the individual's nostrils are flaring (e.g., via use of at least one camera), then it may be determined that the individual is experiencing the emotion 3304 of disgust. And if the biometric data 3302 indicates that the individual's voice is shaky (e.g., via use of at least one microphone), then it may be determined that the individual is experiencing the emotion 3304 of fear.

Information that correlates different biometric data to different emotions or the like can come from different sources. For example, the information could be based on laboratory results, self-reporting trials, and secondary knowledge of emotions (e.g., the individual's use of emoticons and/or words in their communications). Because some information is more reliable than other information, certain information may be weighted more heavily than other information. For example, in certain embodiments, clinical data is weighted heavier than self-reported data. In other embodiments, self-reported data is weighted heavier than clinical data. Laboratory (or learned) results may include data from artificial neural networks, C4.5, classification and/or regression trees, decision trees, deep learning, dimensionality reduction, elastic nets, ensemble learning, expectation maximization, k-means, k-nearest neighbor, kernel density estimation, kernel principle components analysis, linear regression, logical regression, matrix factorization, naïve bayes, neighbor techniques, partial least squares regression, random forest, ridge regression, support vector machines, multiple regression and/or all other learning techniques generally known to those skilled in the art.

Self-reported data may include data where an individual identifies their current state, allowing biometric data to be customized for that individual. For example, computational linguistics could be used to identify not only what an individual is saying but how they are saying it. In other words, the present invention could be used to analyze and chart speech patterns associated with an individual (e.g., allowing the invention to determine who is speaking) and speech patterns associated with how the individual is feeling. For example, in response to “how are you feeling today,” the user may state “right now I am happy,” or “right now I am sad.” Computational linguistics could be used to chart differences in the individual's voice depending on the individual's current emotional state, mood, physical state, or mental state. Because this data may vary from individual to individual, it is a form of self-reported data, and referred to herein as personalized artificial intelligence. The accuracy of such data, learned about the individual's state through analysis of the individual's voice (and then through comparison both to the system's historical knowledge base of states of the individual acquired and stored over time and to a potential wider database of other users' states as defined by analysis of their voice), can further be corroborated and or improved, through cross-referencing the individual's self-reported data with other biometric data, such as heart rate data, etc., when a particular state is self-reported and detected and recorded by the system onto its state profile database.

The collected data, which is essentially a speech/mood profile for the individual (a form of ID which is essentially the individual's unique state profile), can be used by the system that gathered the biometric data or shared with other systems (e.g., the individual's smartphone, the individual's automobile, a voice or otherwise biometrically-enabled device or appliance (including Internet of Things (IOT) devices or IOT system control devices), Internet or “cloud” storage, or any other voice or otherwise biometrically-enabled computing or robotic device or computer operating system with the capability of interaction with the individual, including but not limited to devices which operate using voice interface systems such as Apple's Siri, Google Assistant, Microsoft Cortana, Amazon's Alexa, and their successor systems). Because the shared information is unique to an individual, and can be used to identify a current state of the individual, it is referred to herein as personalized artificial intelligence ID, or “PAIID.” In one embodiment of the present invention, the self-reported data can be thought of as calibration data, or data that can be used to check, adjust, or correlate certain speech patterns of an individual with at least one state (e.g., at least one emotion, at least one mood, at least one physical state, or at least one mental state). The knowledge and predictive nature inherent in the PAIID will be continuously improved through the application of deep learning methodology with data labelling and regression as well as other techniques apparent to those skilled in the art.

With respect to computational linguistics, it should be appreciated that the present invention goes beyond using simple voice analysis to identify a specific individual or what the individual is saying. Instead, the present invention can use computational linguistics to analyze how the individual is audibly expressing himself/herself to detect and determine at least one state, and use this determination as an element in providing content to the user or in performing at least one action (e.g., an action requested by the user, etc.).

It should be appreciated that the present invention is not limited to using a single physical or biological feature (e.g., one set of biometric data) to determine the individual's state. Thus, for example, eye dilation, facial expressions, and heart rate could be used to determine that the individual is surprised. It should also be appreciated that an individual may experience more than one state at a time, and that the received biometric data could be used to identify more than one state, and a system could use their analysis of the individual's state or combination of states to assist it in deciding how best to respond, for example, to a user request, or a user instruction, or indeed whether to do so at all. It should further be appreciated that the present invention is not limited to the six emotions listed in FIG. 32 (i.e., happiness, anger, surprise, sadness, disgust, and fear), and could be used to identify other (or alternate) emotional states, such as regret, love, anxiousness, etc. Finally, the present invention is not limited to the application 3208 as shown in FIG. 31, and may include one or more applications operating on the Web host 3102 and/or the network device 3106. For example, an application or program operating on the network device 3106 could use the biometric data to determine the individual's emotional state, with the emotional state being communicated to the Web host 3102 via the WAN 3100.

Despite preferred embodiments, the present invention is not limited to the use of biometric data (e.g., gathered using sensors, microphones, and/or cameras) solely to determine an individual's current emotional state or mood. For example, an individual's speech (either alone or in combination with other biometric data, such as the individual's blood pressure, heart rate, etc.) could be used to determine the individual's current physical and/or mental health. Examples of physical health include how an individual feels, such as healthy, good, poor, tired, exhausted, sore, achy, and sick (including symptoms thereof, such as fever, headache, sore throat, congested, etc.), and examples of mental health include mental states, such as clear-headed, tired, confused, dizzy, lethargic, disoriented, and intoxicated. By way of example, computational linguistics could be used to correlate speech patterns to at least one physical and/or mental state. This can be done using either self-reported data (e.g., analyzing an individual's speech when the individual states that they are feeling fine, under the weather, confused, etc.), general data that links such biometric data to physical and/or mental state (e.g., data that correlates speech patterns (in general) to at least one physical and/or mental states), or a combination thereof. Such a system could be used, for example, in a hospital to determine a patient's current physical and/or mental state, and provide additional information outside the standard physiological or biometric markers currently utilized in patient or hospital care. If the physical and/or mental state is above/below normal (N), which may include a certain tolerance (T) in either direction (e.g., N+/−T) through the patient making a request or statement, or through response to a question generated by the system, a nurse or other medical staff member may be notified. This would have benefits such as providing an additional level of patient observation automation or providing early warning alerts or reassurance about the patient through system analysis of their state.

As shown in FIG. 31, the Web host 3102 may also include other components, such as a keyboard 3210, allowing a user to enter data, a display 3206, allowing the Web host 3102 to display information to the user (or individual in embodiments where the biometric sensors are internal to the Web Host 3102), a transceiver 3212, allowing the Web host 3102 to communicate with external devices (e.g., the network device 3106 via the WAN 3100, the network device 3106 via a wireless protocol, an external biometric sensor via a wireless protocol, etc.), and a processor 3202, which may control the reception and/or transmission of information to internal and/or external devices and/or run the application 3208, or machine-readable instructions related thereto.

In one embodiment of the present invention, a source of web-based data (e.g., content provider) may express interest in providing the web-based data to an individual in a particular emotional state. For example, as shown in FIG. 33, an owner of feel-good content (e.g., kittens in humorous situations, etc.) may express an interest in providing the content to individuals who are currently feeling the emotion of sadness. The interest may be as simple as “Yes” or “No,” or may be more complex, like interest on a scale of 1-10. In another embodiment of the present invention, a source of web-based data may express interest in providing the web-based data to an individual that experienced a particular emotion in response to a thing (e.g., a person, a place, a subject matter of textual data, a subject matter of video data, a subject matter of audio data, etc.). For example, as shown in FIG. 33, an owner of a matchmaking service may express an interest ($2.50 CPM) in providing a related advertisement to individuals, their friends, or their contacts that experienced the emotion of happiness when they are in close proximity to a wedding (thing) (e.g., being at a wedding chapel, reading an email about a wedding, seeing a wedding video, etc.). By way of another example, an owner of a jewelry store may express an interest (5.00 CPC) in providing an advertisement to individuals that experienced the emotion of excitement when they are in close proximity to a diamond (thing) (e.g., being at a store that sells diamonds, reading an email about diamonds, etc. The selection of web-based content and/or interest may also be based on other data (e.g., demographic data, profile data, click-through responses, etc.). Again, the interest may be a simple “Yes” or “No,” or may be more complex, like an interest on a scale of 1-10, an amount an owner/source of the content is willing to pay per impression (CPM), or an amount an owner/source of the content is willing to pay per click (CPC).

Another embodiment of the invention may involve a system integrated with at least one assistance system, such as voice controls or biometric-security systems, where the emotionally selected messages are primarily warnings or safety suggestions, and are only advertisements in specific relevant situations (discussed in more detail below). An example would be of a user who is using a speech recognition system to receive driving directions where the user's pulse and voice data indicate anger. In this case, the invention may tailor results to be nearby calming places and may even deliver a mild warning that accidents are more common for agitated drivers. This is an example where the primary purpose of the use is not the detection of emotion, but the emotion data can be gleaned from such systems and used to target messages to the individual, contacts, care-providers, employers, or even other computer systems that subscribe to emotional content data. An alternate example would be a security system that uses retinal scanning to identify pulse and blood pressure. If the biometric data correlates to sadness, the system could target the individual with uplifting or positive messages to their connected communication device or even alert a care-provider. In other instances, for example with a vehicle equipped with an autonomous driving system, based on the system's analysis of the biometric feedback of the individual, the driving system could advise on exercising caution or taking other action in the interests of the driver and others (e.g., passengers, drivers of other vehicles, etc.).

It should be noted that in this invention some use cases the individual's private data is provided with the users consent to the system, but in many cases the emotional response could be associated with a time-of-day, a place, or a given thing (e.g., jewelry shop, etc.), so personally identifying information (PII) does not need to be shared with the message provider. In the example of a jewelry shop, the system simply targets individuals and their friends with strong joy correlations. While in certain embodiments, individuals may be offered the opportunity to share their PII with message providers, the system can function without this level of information.

The interest data, and perhaps other data (e.g., randomness, demographics, etc.) may be used by the application (FIG. 31 at 3208) to determine web-based data (e.g., an advertisement, etc.) that should be provided to the individual. For example, if the interest data includes different bids for a particular emotion or an emotion-thing relationship, the application may provide the advertisement associated with the highest bid to the individual (or related network device) who experienced the emotion. In other embodiments, other data is taken into consideration in providing web-based data to the individual. In these embodiments, interest data is but one criteria that is taken into account in selecting web-based data that is provided to the individual.

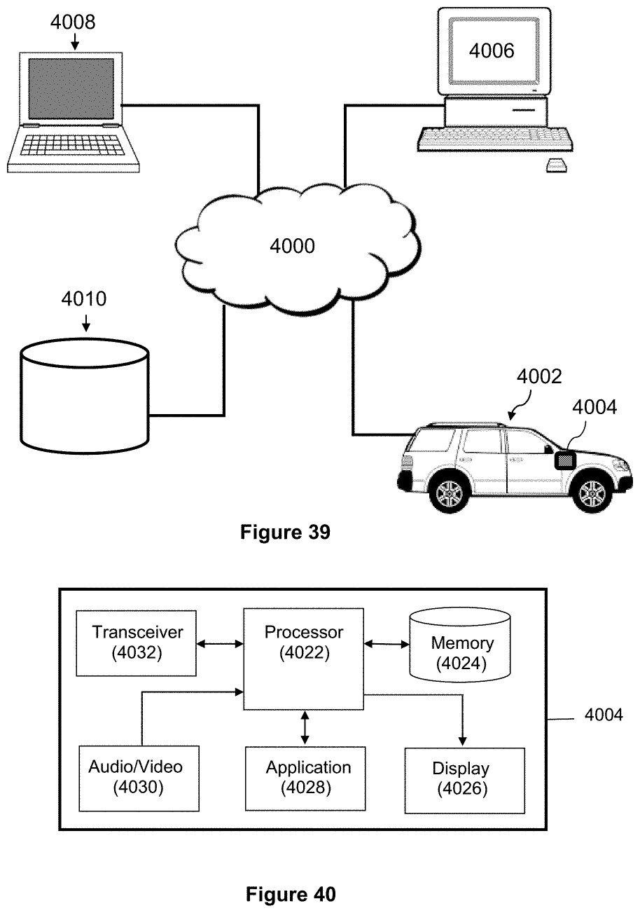

It should be appreciated that the “response” to an individual in a particular state, or having an emotional response to a thing, is not limited to providing the individual with web-based content, and may include any action consistent with the determined state. In other words, the determined state can be used by the host (e.g., automobile, smartphone, etc.) to determine context, referred to herein as “situational context.” For example, as shown in FIG. 39, an automobile 4002 may include a host 4004 that determines (using biometric data acquired via a camera, microphone, or sensor) that the driver (not shown) is impaired or emotional (e.g., angry, excited, etc.), may switch to auto-pilot, or may limit the maximum speed of the vehicle. In this embodiment, the “response” carried out by the host may be based on commands provided by the individual (e.g., verbal or otherwise) and at least one emotion or mood of the individual, where the emotion/mood is determined based on biometric data. For example, where a voice command to perform an action (by itself) may result in a robot performing an action at a normal pace (which may have the benefit of battery preservation, accuracy, etc.), a voice command to perform the same action along with biometric data expressing a mood of urgency may result in the robot performing the action at a quicker pace.

In one embodiment of the present invention, the host 4004 is a network-enabled device and is configured to communicate with at least one remote device (e.g., 4006, 4008, 4010) via a wide area network (WAN) 4000. For example, the host 4004 may be configured to store/retrieve individual state profiles (e.g., PAIID) on/from a remote database (e.g., a “cloud”) 4010, and/or share individual state profiles (e.g., PAIID) with other network-enabled devices (e.g., 4006, 4008). The profiles could be stored for future retrieval, or shared in order to allow other devices to determine an individual's current state. As discussed above, the host 4004 may gather self-reporting data that links characteristics of the individual to particular states. By sharing this data with other devices, those devices can more readily determine the individual's current state without having to gather (from the individual) self-reporting (or calibration) data. The database 4010 could also be used to store historical states, or states of the individual over a period of time (e.g., a historical log of the individual's prior states). The log could then be used, either alone or in conjunction with other data, to determine an individual's state during a relevant time or time period (e.g., when the individual was gaining weight, at the time of an accident, when performing a discrete or specific action, etc.), or to determine indications as to psychological aptitude or fitness to perform certain functions where, for example an individual's state is of critical importance, such as, but not limited to piloting a plane, driving a heavy goods' vehicle, or trading instructions on financial or commodities exchanges.

The state log could be further utilized to generate a state “bot” which is an agent of the individual capable of being distributed over a network to look for information on behalf of the individual which is linked to a particular thing the individual has an “interest” in, or wishes to be informed of, either positive or negative, conditional on their being in that particular state.

In an alternate embodiment, information, such as historical logs or individual state profiles (e.g., PAIID) are also, or alternatively, stored on a memory device 4024 on the host 4004 (see FIG. 40). In this embodiment, the host 4004 may include a transceiver 4032, a processor 4022, a display 4026, and at least one application 4028 (see FIG. 40), all of which function the same as similar components depicted in FIG. 31. The host 4004 may also include at least one microphone and/or at least one camera 4030 configured to acquire audio/video from/of the individual (e.g., a driver of a vehicle). As previously discussed, the audio/video can be used to determine at least one state of the individual. For example, the individual's speech and/or facial features, either alone or in combination with other data (e.g., heart rate data acquired from sensors on the steering wheel, etc.), could be analyzed to determine at least one state of the individual. The state can then be used to perform at least one action. In one embodiment of the present invention, the state is used to determine whether a request (e.g., command, etc.) from the individual should be carried out, and if so, whether other actions should also be performed (e.g., limiting speed, providing a warning, etc.). For example, if a driver of a vehicle instructs the vehicle to start, the vehicle (or host operating therein) could provide the driver with a warning if it is determined that the driver is tired, or could initiate auto-pilot mode if it is determined that the driver is impaired (e.g., under the influence). In another example, an airline pilot could be asked to provide a response as to how they're feeling, and dependent on how the pilot responds, both by nature of the content of their reply and its analyzed state, air traffic control can seek to take the appropriate action to seek to ensure the safety of the plane. In this case, and cases of a similar nature or context failure to provide any kind of response would provide an alert which might indicate either that the pilot didn't wish to respond (which is information in itself) or was not in a situation to respond.

It should be appreciated that in embodiments where the individual is responding to a thing, the thing could be anything in close proximity to the individual, including a person (or a person's device (e.g., smartphone, etc.)), a place (e.g., based on GPS coordinates, etc.), or content shown to the user (e.g., subject matter of textual data like an email, chat message, text message, or web page, words included in textual data like an email, chat message, text message, or web page, subject matter of video data, subject matter of audio data, etc.). The “thing” or data related thereto can either be provided by the network device to the Web host, or may already be known to the Web host (e.g., when the individual is responding to web-based content provided by the Web host, the emotional response thereto could trigger additional data, such as an advertisement).

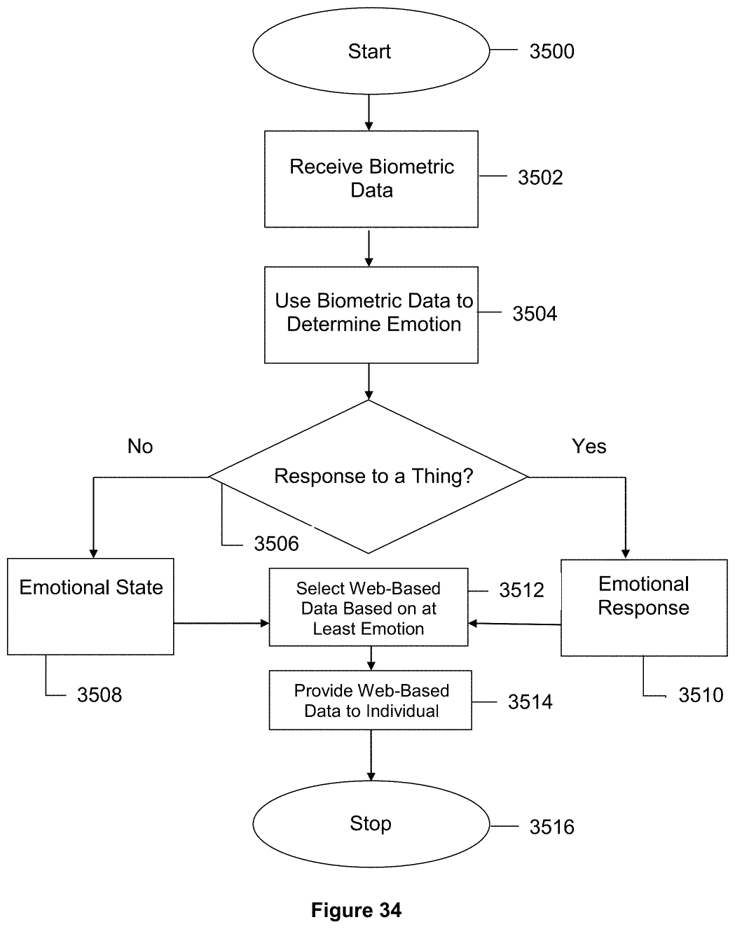

A method of carrying out the present invention, in accordance with one embodiment of the present invention, is shown in FIG. 34. Starting at step 3500, biometric data is received at step 3502. As discussed above, the biometric data can be at least one physical and/or biological characteristics of an individual, including, but not limited to, heart rate, blood pressure, temperature, breathing rate, facial features, changes in speech, changes in eye movement and/or dilation, and chemical compositions (in blood, sweat, saliva, urine or breath). The biometric data is then used to determine a corresponding emotion at step 3504, such as happiness, anger, surprise, sadness, disgust, or fear. At step 3506 a determination is made as to whether the emotion is the individual's current state, or whether it is based on the individual's response to a thing (e.g., a person, place, information displayed to the individual, etc.). If the emotion is the individual's current state (step 3508), then web-based data is selected based on the individual's current emotional state at step 3512. If, however, the emotion is the individual's response to a thing (step 3510), then web-based data is selected based on the individual's emotional response to the thing at step 3510. The selected web-based data is then provided to the individual at step 3514, stopping the process at step 3516.

It should be appreciated that the present invention is not limited to the method shown in FIG. 34, and methods that includes additional, fewer, or different steps is within the spirit and scope of the present invention. For example, at step 3512, the web-based data may be selected using emotion data (or emotion-thing data) and interest data. By way of another example, in step 3514, the selected content (e.g., web-based data, text message, email, etc.) may also (or alternatively) be provided to a third person, such as a legal guardian of the individual, a family member of the individual, a medical staff member (if the individual is in the hospital), emergency response (if the individual is not in the hospital), etc. The present invention is also not limited to the steps recited in FIG. 34 being performed in any particular order. For example, determining whether the emotion is the individual's current state or the individual's response to a thing may be performed before the reception of biometric data.

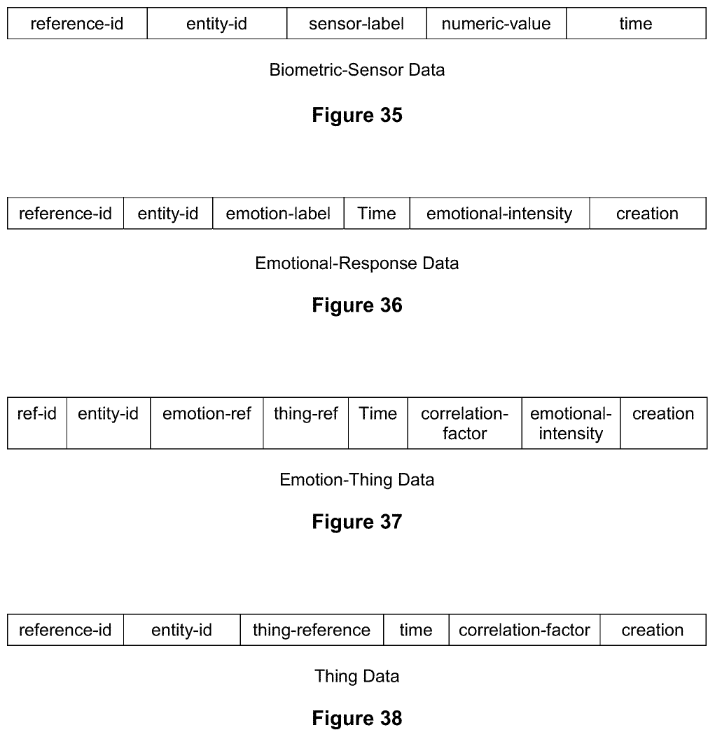

While biometric data, and the like, can be very simple in nature (e.g., identifying the characteristic being measured, such as blood pressure, and the measured value, such as 120/80), it can also be quite complex, allowing for data to be stored for subsequent use (e.g., creating profiles, charts, etc.). For example, in one embodiment of the present invention, as shown in FIG. 35, biometric-sensor data may include detailed data, such as reference-id (technical unique-identify of this datum), entity-id (a user, team, place word or number, device-id), sensor-label (a string describing what is being measured), numeric-value (integer or float), and/or time (e.g., GMT UNIX of when the measurement was taken). As shown in FIG. 36, emotional-response data may include reference-id (technical unique-identifier of this datum), entity-id (a user, team, place word or number, device-id), emotion-label (a string that recognizes this as an emotion), time (e.g., GMT UNIX timestamp when this record was created), emotional-intensity (numeric-value), and/or datum-creation data (a technical reference to what system created this datum and/or which data was used to create this datum). As shown in FIG. 37, emotion-thing data may include reference-id (technical unique-identifier of this datum), entity-id (a user, team, place word or number, device-id), emotion-reference (a reference to a specific emotion documented elsewhere), thing-reference (a reference to a specific thing documented elsewhere), time (e.g., GMT UNIX timestamp when this record was created), correlation-factor (numeric-value representing a scale of correlation, such as a percent), emotional-intensity (numeric-value), and/or datum-creation data (a technical reference to both what system created this datum and/or which data was used to create this datum). As shown in FIG. 38, thing data may include reference-id (technical unique-identifier of this datum), entity-id (a user, team, place word or number, device-id), thing-reference (a reference to specific “thing” documented elsewhere), time (e.g., GMT UNIX timestamp when this records was created), correlation-factor (numeric-value representing a scale of correlation, such as a percent), and/or datum-creation data (a technical reference to both what system created this datum and/or which data was used to create this datum). It should be appreciated that the present invention is not limited to the data strings shown in FIGS. 35-38, and other methods of communicating said data is within the spirit and scope of the present invention.

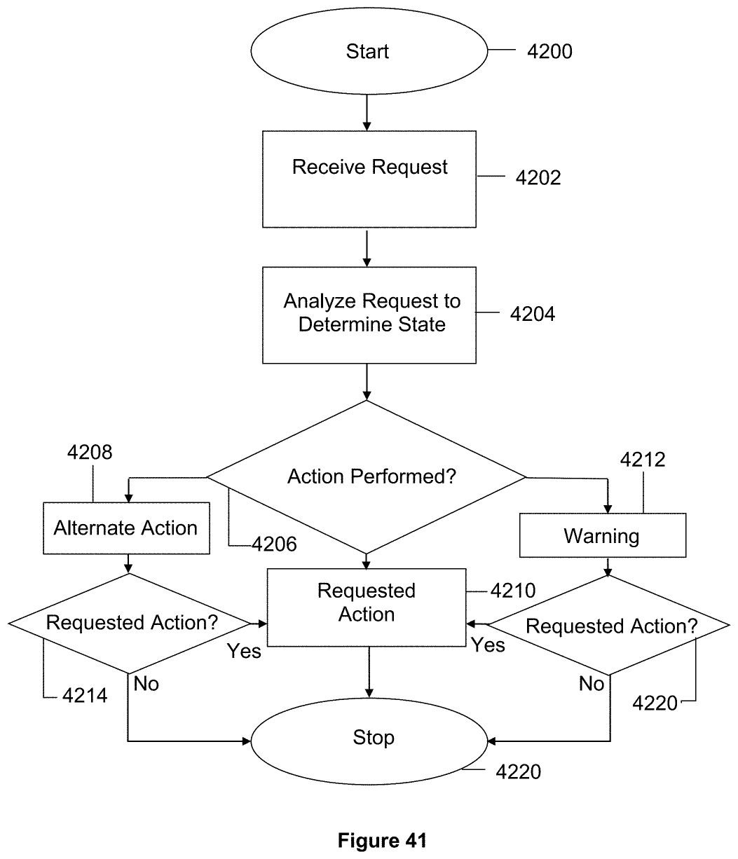

A method of carrying out the present invention, in accordance with another embodiment of the present invention, is shown in FIG. 41. Starting at step 4200, a request is received from a user at step 4202. As discussed above, the request may include a question asked by the user (dictating a response) or a command provided by the user (dictating the performance of an action). The request (or other biometric data) is then analyzed to determine the user's current state at step 4204, such as a corresponding emotional state, mood, physical state, and/or mental state. At step 4206, the user's current state is used to determination whether a particular action should be performed. For example, if the user's state is normal, then the requested action (e.g., the action requested at step 4202) is performed at step 4210, ending the method at step 4220. If the user's state is abnormal, but not alarming (e.g., angry), then a warning may be provided at step 4212. If the user's state is abnormal and alarming (e.g., intoxicated), then a different action (e.g., an action that is different from the one requested at step 4202) may be performed at step 4208. If a warning is provided at step 4212, or a different action is performed at step 4208, then a determination is made at steps 4220 and 4214, respectively, as to whether the requested action (e.g., the action requested at step 4202) should be performed. If the answer is YES, then the requested action is performed at step 4210, ending the method at step 4220. If the answer is NO, then no further action is taken, ending the method at step 4220.

It should be appreciated that the present invention is not limited to the method shown in FIG. 41, and methods that includes additional, fewer, or different steps is within the spirit and scope of the present invention. The present invention is also not limited to the steps recited in FIG. 41 being performed in any particular order.

The foregoing description of a system and method for using at least self-reporting and biometric data to determine a current state of a user has been presented for the purposes of illustration and description. It is not intended to be exhaustive or to limit the invention to the precise forms disclosed, and many modifications and variations are possible in light of the above teachings. Those skilled in the art will appreciate that there are a number of ways to implement the foregoing features, and that the present invention it not limited to any particular way of implementing these features. The invention is solely defined by the following claims.

What is claimed is:

1. A system that uses artificial intelligence (AI) to determine a state of a user, said determined state comprising one of an emotional and physical state, comprising:
at least one server in communication with a wide area network (WAN); and
at least one memory device for storing machine readable instructions, at least a first set of said machine readable instructions being provided to a mobile device via said at least one server and said WAN, said first set of said machine readable instructions being adapted to operate on said mobile device and perform the steps of:
providing first content to said user;
receiving first biometric data of said user, said first biometric data being received from at least one sensor and comprising at least a change in said user's pupil in response to said first content;
receiving self-reporting data from said user, said self-reporting data being received after said first biometric data and comprising a first state of said user;
storing said first biometric data and said self-reporting data in a memory, said first biometric data being linked to said self-reporting data;
providing second content to said user;
receiving second biometric data from said user, said second biometric being received from said at least one sensor after said self-reporting data and in response to said second biometric data; and
using at least said first biometric data, said second biometric data, and said self-reporting data to determine a second state of said user at a time that said second biometric data is received.

2. The system of claim 1, wherein at least said first content comprises varying visuals provided to said user via a display on said mobile device.

3. The system of claim 2, wherein said at least one sensor comprises at least one camera on said mobile device.

4. The system of claim 3, wherein said at least one camera on said mobile device is used to capture changes in said user's pupil from a dilated state in response to changes from a first visual to a second visual provided by said display on said mobile device.

5. The system of claim 1, wherein said emotional state comprises at least one of happiness, sadness, surprise, anger, disgust, fear, euphoria, attraction, love, arousal, calmness, amusement, excitement, tiredness, hunger, thirst, well-being, sick, failure, triumph, interest, enthusiasm, animation, reinvigoration, and satisfaction.

6. The system of claim 4, wherein said at least one camera is further used to acquire heart data of said user, said heart data being used together with at least said first biometric data, said second biometric data, and said self-reporting data to determine said second state.

7. The system of claim 6, wherein said heart data comprises heart rate variability (HRV) received from a second sensor in communication with said mobile device.

8. The system of claim 6, wherein said heart data comprises at least one of pulse and blood pressure and is received using said camera on said mobile device.

9. The system of claim 1, wherein said changes in said user pupil further comprises movement of said pupil.

10. The system of claim 1, wherein said machine readable instructions are further adapted to perform the step of performing at least one action in response to said determined second state.

11. The system of claim 3, wherein said machine readable instructions are further adapted to perform the step of receiving at least ambient data, said ambient data being used in said step of determined said second state.

12. A method for using artificial intelligence (AI) to determine a state of a user, said determined state comprising one of an emotional and physical state, comprising:
providing first content to said user;
receiving first biometric data of said user, said first biometric data being received from at least one sensor and comprising at least a change in said user's pupil in response to said first content;
receiving self-reporting data from said user, said self-reporting data being received after said first biometric data and comprising a first state of said user;
storing said first biometric data and said self-reporting data in a memory, said first biometric data being linked to said self-reporting data;
providing second content to said user;
receiving second biometric data from said user, said second biometric being received from said at least one sensor after said self-reporting data and in response to said second biometric data; and
using at least said first biometric data, said second biometric data, and said self-reporting data to determine a second state of said user at a time that said second biometric data is received.

13. The method of claim 12, wherein at least said first content comprises varying visuals provided to said user via a display on a mobile device.

14. The method of claim 13, wherein said at least one sensor comprises a camera on said mobile device.

15. The method of claim 14, wherein said camera on said mobile device is used to capture changes in said user's pupil from a dilated state in response to changes from a first visual to a second visual provided by said display on said mobile device.

16. The method of claim 14, wherein said step of using at least said first biometric data, said second biometric data, and said self-reporting data to determine a second state further comprises using at least said first biometric data, said second biometric data, said self-reporting data, and heart data to determine said second state.

17. The method of claim 16, wherein said heart data comprises heart rate variability (HRV) received from a second sensor in communication with said mobile device.

18. The method of claim 16, wherein said heart date comprises at least one of pulse and blood pressure and is received using said camera on said mobile device.

19. A method for using artificial intelligence (AI) to determine a state of a user, comprising:
using a display on a mobile device to provide first visual content to said user;
receiving first biometric data of said user, said first biometric data being received from at least a camera on said mobile device and comprising various levels of dilation of said user's pupil responsive to said first visual content;
receiving self-reporting data from said user, said self-reporting data being received after said first biometric data and comprising a first state of said user;
storing said first biometric data and said self-reporting data in a memory, said first biometric data being linked to said self-reporting data;
using said display on said mobile device to provide second visual content to said user;
receiving second biometric data from said user, said second biometric being received from at least said camera on said mobile device after said self-reporting data and in response to said second biometric data;
using at least said first biometric data, said second biometric data, and said self-reporting data to determine a second state of said user at a time that said second biometric data is received;
performing at least one action in response to said second state.

20. The method of claim 19, wherein said at least one action is reporting said second state to said user.
